(19) United States
(12) Patent Application Publication
(10) Pub. No.: US 2022/0054448 A1
(43) Pub. Date: Feb. 24, 2022

(54) NOVEL POLYMETHOXYFLAVONE COMPOUNDS FOR SKELETAL MUSCLE MODULATION, METHODS AND USES THEREOF

(71) Applicant: SOCIETE DES PRODUITS NESTLE S.A., Vevey (CH)

(72) Inventors: Joris Michaud, Lausanne (CH); Jerome Feige, Crissier (CH); Pascal Stuelsatz, Crissier (CH); Sonia Karaz, Epalinges (CH); Denis Marcel Barron, Lutry (CH); Yann Ratinaud, Morges (CH); Benjamin Brinon, Mex (CH)

(21) Appl. No.: 17/416,211

(22) PCT Filed: Dec. 17, 2019
(86) PCT No.: PCT/EP2019/085526

§ 371 Date: Jun. 18, 2021

(30) Foreign Application Priority Data
Dec. 20, 2018 (EP) 18214385.9

(51) Int. Cl. A61K 31/352; A61P 21/06

(52) U.S. Cl. CPC A61K 31/352; A61P 21/06

(57) ABSTRACT

The present invention relates to novel polymethoxyflavone compounds for improving skeletal muscle plasticity regeneration to maintain or increase muscle function and/or muscle mass by modulating muscle stem cells. For example, the present invention is useful for subjects to promote muscle repair and/or subjects suffering from precachexia, cachexia, sarcopenia, myopathy, dystrophy and/or recovery after muscle injury or surgery.

### FIELD OF THE INVENTION

The present invention relates to novel polymethoxyflavone compounds for improving skeletal muscle plasticity to maintain or increase muscle function and/or muscle mass by modulating muscle stem cells. For example, the present invention is useful for subjects to promote muscle repair and/or subjects suffering from precachexia, cachexia, sarcopenia, myopathy, dystrophy and/or recovery after muscle injury or surgery.

### BACKGROUND TO THE INVENTION

Skeletal muscle regeneration is a crucial mechanism to repair and maintain muscle mass and function throughout life. Skeletal muscle regeneration primarily requires the participation of myogenic progenitors, known as muscle stem cells or satellite cells.

Non-proliferative, quiescent satellite cells, which adjoin resting skeletal muscles, can be identified by their distinct location between sarcolemma and basal lamina, a high nuclear-to-cytoplasmic volume ratio, few organelles (e.g. ribosomes, endoplasmic reticulum, mitochondria, golgi complexes), small nuclear size, and a large quantity of nuclear heterochromatin relative to myonuclei. On the other hand, activated satellite cells have an increased number of caveolae, cytoplasmic organelles, and decreased levels of heterochromatin.

These muscle satellite cells are part of the adult stem cell niche and they are involved in the normal growth of muscle, as well as regeneration following injury or disease. Hence, they are a potential target to enhance muscle regeneration in both healthy and diseased conditions. Skeletal muscle regeneration follows a series of steps that recapitulates the phases of development. Muscle progenitor cells must exit the state of quiescence and become active, proliferate and commit to myogenic differentiation.

Satellite cells express genetic markers at different stages of myogenesis and proliferation. Pax7 and Pax3 are considered to be satellite cell markers. For example, activated satellite cells expressing low levels of Pax7 are more committed to differentiation, whereas high levels of Pax7 are related to cells less prone to differentiate and have more undifferentiated stemness characteristics. Activation and the induction of myogenesis is typically regulated by myogenic regulatory factors such as MyoD, Myf5, myogenin and MRF4. Negative regulation by myostatin and TGFb inhibits the differentiation of satellite cells (Almeida et al., 2016).

Experimental therapies which have previously included myoblast transplantation have not been entirely successful due to the reduced regenerative potential of myoblasts which are more committed and differentiated in comparison the muscle stem cells.

Therefore, there remains a significant need to identify compounds, compositions and methods which modulate muscle stem cells directly for maintaining muscle health and improving muscle regeneration. Such compounds, compositions and methods of treatment may help subjects with muscle stem cell dysfunction and/or subjects suffering from muscular diseases and conditions, such as cachexia or sarcopenia by facilitating maintenance of, increasing muscle function and/or muscle mass.

### SUMMARY OF THE INVENTION

The present invention has identified novel polymethoxyflavone compounds and compositions to modulate skeletal muscle function and improve skeletal muscle regeneration in order improve muscle repair after injury or to counteract muscle wasting that occurs in a number of pathological conditions, in particular, cachexia and sarcopenia.

In one embodiment, the present invention relates to a compound of general formula (I):

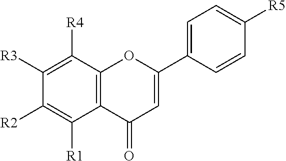

wherein R1, R2, R3, and R4, are each independently H; OH; OMe; O-glycoside; C-glycoside; acylated O-glycoside; acylated C-glycoside; sulfated O-glycoside; sulfated C-glycoside; a halogen; a ketone; an aldehyde; a carboxylic acid; an ester; a primary, secondary, or tertiary amine; a primary or secondary amide; a cyano; a nitro; a sulfonate; a sulfate; an optionally substituted and/or optionally branched C1 to C20 alkyl; an optionally substituted and/or optionally branched, C2 to C20 alkenyl; an optionally substituted and/or optionally branched, C4 to C20 polyalkenyl; an optionally substituted and/or optionally branched C2 to C20 alkynyl, or an optionally substituted and/or optionally branched C4 to C20 polyalkynyl and R5 is H; OH; O-glycoside; C-glycoside; acylated O-glycoside; acylated C-glycoside; sulfated O-glycoside; sulfated C-glycoside; a halogen; a ketone; an aldehyde; a carboxylic acid; an ester; a primary, secondary, or tertiary amine; a primary or secondary amide; a cyano; a nitro; a sulfonate; a sulfate; an optionally substituted and/or optionally branched C1 to C20 alkyl; an optionally substituted and/or optionally branched, C2 to C20 alkenyl; an optionally substituted and/or optionally branched, C4 to C20 polyalkenyl; an optionally substituted and/or optionally branched C2 to C20 alkynyl, or an optionally substituted and/or optionally branched C4 to C20 polyalkynyl.

In some embodiments, a OMe group can cyclize with a neighboring OH group to form a methylene dioxy bridge.

In another embodiment, the invention relates to compounds of general formula (II):

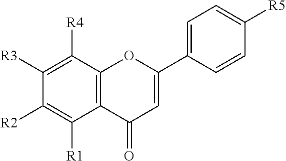

wherein R1, R2, R3, and R4 are each independently H; OH; OMe; O-glycoside; a sulfate; an optionally substituted and/or optionally branched C1 to C20 alkyl; an optionally substituted and/or optionally branched, C2 to C20 alkenyl; or an optionally substituted and/or optionally branched, C4 to C20 polyalkenyl and R5 is H; OH; O-glycoside; a sulfate; an optionally substituted and/or optionally branched C1 to C20 alkyl; an optionally substituted and/or optionally branched, C2 to C20 alkenyl; or an optionally substituted and/or optionally branched, C4 to C20 polyalkenyl.

In some embodiments, a OMe group can cyclize with a neighboring OH group to form a methylene dioxy bridge.

In another embodiment, the invention relates to compounds of general formula (III):

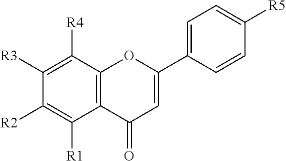

wherein R1, R2, R3, and R4 are each independently H; OH; OMe; O-glycoside; or a sulfate and R5 is H; OH; O-glycoside; or a sulfate.

In some embodiments, a OMe group can cyclize with a neighboring OH group to form a methylene dioxy bridge.

In one embodiment, said compound is Xanthomicrol (5,4′-Dihydroxy-6,7,8-trimethoxyflavone, CAS number 16545-23-6):

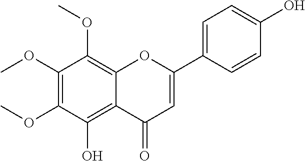 [SMILES: COc1c(OC)c(O)c2c(=O)cc(-c3ccc(O)cc3)oc2c1OC]

Xanthomicrol is also known as; 4H-1-Benzopyran-4-one, 5-hydroxy-2-(4-hydroxyphenyl)-6,7,8-trimethoxy; Flavone 4′,5-dihydroxy-6,7,8-trimethoxy; 5-Hydroxy-2-(4-hydroxyphenyl)-6,7,8-trimethoxy-4H-1-benzopyran-4-one; and NSC 79323 with molecular formula C₁₈H₁₆O₇ and molecular weight 344.32.

In one embodiment, said compound is Cirsimaritin (5,4′-Dihydroxy-6,7-dimethoxyflavone, CAS number 6601-62-3):

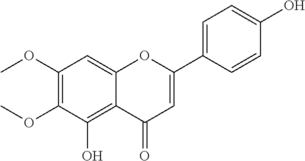 [SMILES: COc1cc2oc(-c3ccc(O)cc3)cc(=O)c2c(O)c1OC]

Cirsimaritin is also known as 4H-1-Benzopyran-4-one, 5-hydroxy-2-(4-hydroxyphenyl)-6,7-dimethoxy; Flavone, 4′,5-dihydroxy-6,7-dimethoxy; 5-Hydroxy-2-(4-hydroxyphenyl)-6,7-dimethoxy-4H-1-benzopyran-4-one; 4′,5-Dihydroxy-6,7-dimethoxyflavone; 5,4′-Dihydroxy-6,7-dimethoxyflavone; 6,7-Dimethoxyscutellarein; 6-Methoxyapigenin 7-methyl ether; 6-Methoxygenkwanin; 7-Methylcapillarisin; Cismaritin; Cirsitakaogenin; Cirsumaritin; Scrophulein; Scutellarein 6,7-dimethyl ether; and Skrofulein with molecular formula C₁₇H₁₄O₆ and molecular weight 314.29.

In one embodiment, said compound is Cirsimarin (5,4′-Dihydroxy-6,7-dimethoxyflavone 4′-O-β-D-glucoside, CAS number 13020-19-4):

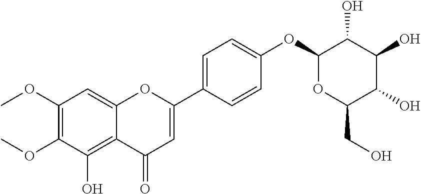 [SMILES: COc1cc2oc(-c3ccc(O[C@@H]4O[C@H](CO)[C@@H](O)[C@H](O)[C@H]4O)cc3)cc(=O)c2c(O)c1OC]

Cirsimarin is also known as 4H-1-Benzopyran-4-one, 2-[4-(β-D-glucopyranosyloxy)phenyl]-5-hydroxy-6,7-dimethoxy; 2-[4-(β-D-Glucopyranosyloxy)phenyl]-5-hydroxy-6,7-dimethoxy-4H-1-benzopyran-4-one; 5,4′-Dihydroxy-6,7-dimethoxyflavone 4′-O-β-D-glucoside; Cirsimaretin; Cirsimarine; Cirsimaritin 4′-glucoside; Cirsimartin; Cirsitakaoside; and Cismaritin with molecular formula C₂₃H₂₄O₁₁ and molecular weight 476.43.

In one embodiment, the invention relates to compounds of general formula (IV)

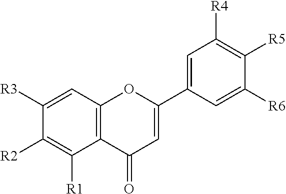

wherein R1, R2, and R3 are each independently H; OH; OMe; O-glycoside; C-glycoside; acylated O-glycoside; acylated C-glycoside; sulfated O-glycoside; sulfated C-glycoside; a halogen; a ketone; an aldehyde; a carboxylic acid; an ester; a primary, secondary, or tertiary amine; a primary or secondary amide; a cyano; a nitro; a sulfonate; a sulfate; an optionally substituted and/or optionally branched C1 to C20 alkyl; an optionally substituted and/or optionally branched, C2 to C20 alkenyl; an optionally substituted and/or optionally branched, C4 to C20 polyalkenyl; an optionally substituted and/or optionally branched C2 to C20 alkynyl, or an optionally substituted and/or optionally branched C4 to C20 polyalkynyl and R4, R5 and R6, are each independently OH; OMe; O-glycoside; C-glycoside; acylated O-glycoside; acylated C-glycoside; sulfated O-glycoside; sulfated C-glycoside; a halogen; a ketone; an aldehyde; a carboxylic acid; an ester; a primary, secondary, or tertiary amine; a primary or secondary amide; a cyano; a nitro; a sulfonate; a sulfate; an optionally substituted and/or optionally branched C1 to C20 alkyl; an optionally substituted and/or optionally branched, C2 to C20 alkenyl; an optionally substituted and/or optionally branched, C4 to C20 polyalkenyl; an optionally substituted and/or optionally branched C2 to C20 alkynyl, or an optionally substituted and/or optionally branched C4 to C20 polyalkynyl.

In some embodiments, a OMe group can cyclize with a neighboring OH group to form a methylene dioxy bridge.

In one embodiment, the invention relates to compounds of general formula (V):

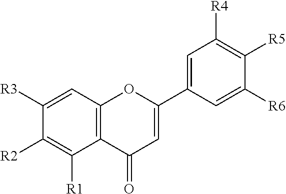

wherein R1, R2 and R3 are each independently H; OH; OMe; O-glycoside; a sulfate; an optionally substituted and/or optionally branched C1 to C20 alkyl; an optionally substituted and/or optionally branched, C2 to C20 alkenyl; or an optionally substituted and/or optionally branched, C4 to C20 polyalkenyl and R4, R5 and R6, are each independently OH; OMe; O-glycoside; a sulfate; an optionally substituted and/or optionally branched C1 to C20 alkyl; an optionally substituted and/or optionally branched, C2 to C20 alkenyl; or an optionally substituted and/or optionally branched, C4 to C20 polyalkenyl.

In some embodiments, a OMe group can cyclize with a neighboring OH group to form a methylene dioxy bridge.

In one embodiment, the invention relates to compounds of general formula (VI):

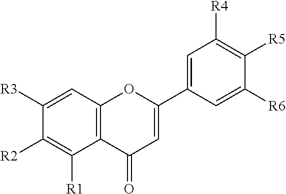

wherein R1, R2 and R3 are each independently H; OH; OMe; O-glycoside; a sulfate and R4, R5 and R6, are each independently OH; OMe; O-glycoside; a sulfate.

In some embodiments, a OMe group can cyclize with a neighboring OH group to form a methylene dioxy bridge.

In one embodiment, said compound is 5,6,7,3′,4′,5′-Hexamethoxyflavone, CAS number 29043-07-0):

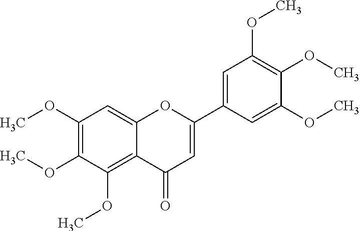

5,6,7,3′,4′,5′-Hexamethoxyflavone is also known as 4H-1-Benzopyran-4-one, 5,6,7-trimethoxy-2-(3,4,5-trimethoxyphenyl); Flavone 3′,4′,5,5′,6,7-hexamethoxy; 5,6,7-Trimethoxy-2-(3,4,5-trimethoxyphenyl)-4H-1-benzopyran-4-one; and 3′,4′,5′,5,6,7-Hexamethoxyflavone with molecular formula C₂₁H₂₂O₈ and molecular weight 402.39.

In one embodiment, the invention relates to compounds of general formula (VII):

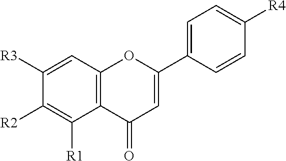

wherein R1 and R2 are each independently H or OH and R3 and R4, are each independently H; OH; OMe; O-glycoside; C-glycoside; acylated O-glycoside; acylated C-glycoside; sulfated O-glycoside; sulfated C-glycoside; a halogen; a ketone; an aldehyde; a carboxylic acid; an ester; a primary, secondary, or tertiary amine; a primary or secondary amide; a cyano; a nitro; a sulfonate; a sulfate; an optionally substituted and/or optionally branched C1 to C20 alkyl; an optionally substituted and/or optionally branched, C2 to C20 alkenyl; an optionally substituted and/or optionally branched, C4 to C20 polyalkenyl; an optionally substituted and/or optionally branched C2 to C20 alkynyl, or an optionally substituted and/or optionally branched C4 to C20 polyalkynyl.

In one embodiment, the invention relates to compounds of general formula (VIII):

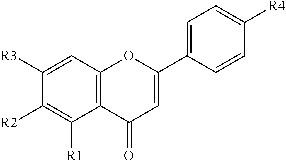

wherein R1 and R2 are each independently H or OH and R3 and R4, are each independently H; OH; OMe; O-glycoside; a sulfate; an optionally substituted and/or optionally branched C1 to C20 alkyl; an optionally substituted and/or optionally branched, C2 to C20 alkenyl; or an optionally substituted and/or optionally branched, C4 to C20 polyalkenyl.

In one embodiment, the invention relates to compounds of general formula (IX):

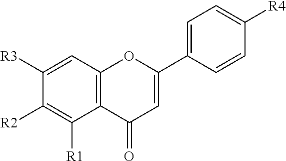

wherein R1 and R2 are each independently H or OH and R3 and R4, are each independently H; OH; OMe; O-glycoside; a sulfate.

In one embodiment, said compound is Ladanein (5,6-Dihydroxy-7,4′-dimethoxyflavone, CAS number 10176-71-3).

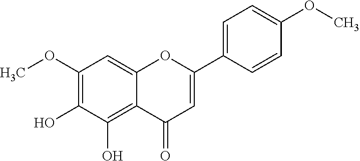

Ladanein is also known as 4H-1-Benzopyran-4-one, 5,6-dihydroxy-7-methoxy-2-(4-methoxyphenyl; Flavone 5,6-dihydroxy-4′,7-dimethoxy; 5,6-Dihydroxy-7-methoxy-2-(4-methoxyphenyl)-4H-1-benzopyran-4-one; 4′,7-Di-O-methylscutellarein; 5,6-Dihydroxy-4′,7-dimethoxyflavone; 5,6-Dihydroxy-7,4′-dimethoxyflavone; BJ 486K; Ladanine; and Scutellarein 4′,7-dimethyl ether with molecular formula C₁₇H₁₄O₆ and molecular weight 314.29.

The compounds and compositions of the present invention, may be useful for modulating muscle stem cell function to maintain or increase skeletal muscle function and/or mass in a subject, and/or substantially prevent or reduce muscle wasting in a subject. In particular, to enhance: the number of muscle stem cells, the function of muscle stem cells, myogenesis and muscle growth.

The compounds and compositions of the present invention, may be useful to promote muscle regeneration, recovery from muscle wasting or muscle injury, and/or to prevent or treat sarcopenia or cachexia; or precachexia. In particular, wherein sarcopenia is loss of muscle mass and/or strength linked to aging and cachexia is associated with a disease, for example, when associated with cancer, chronic heart failure, renal failure, chronic obstructive pulmonary disease, AIDS, autoimmune disorders, chronic inflammatory disorders, cirrhosis of the liver, anorexia, chronic pancreatitis, metabolic acidosis and/or neurodegenerative disease (Von Haehling et al. 2014).

The compounds and compositions of the present invention may be useful to promote muscle mass and muscle function in a non-human animal for optimizing meat production.

### DESCRIPTION OF THE DRAWINGS

FIGS. 1 to 3—Myogenic Commitment of Muscle Stem Cells

Human primary myoblasts from two different donors (donor 8 & donor 4) were seeded in 384 well plates at a density of 1,000 cells per well in skeletal muscle growth medium (SKM-M, AMSbio). For treatment, compounds were directly added to the myoblast cultures 16 hours after initial plating.

FIG. 1 represents the compound xanthomicrol

FIG. 2 represents the compound cirsimaritin

FIG. 3 represents the compound cirsimarin

All cultures were then grown for 96 hours. Cells were stained for Pax7 and MyoD expression using antibodies directed against Pax7 and MyoD and counterstained with Hoechst 33342 to visualize cell nuclei. Myoblasts (MyoD+) are defined as cells that do not express Pax7 but express MyoD. Image acquisition was performed using the ImageXpress (Molecular Devices) platform. Custom module analysis based on Multi-Wavelength Cell Scoring of the MetaXpress software was used for quantification. For each condition, the total number of cells was determined to evaluate compound toxicity, and the number of MyoD+ cells was normalized to the total cell number in order to evaluate the proportion of this population. *, **, ***, **** indicates difference from the control, One-way ANOVA, with p<0.05, p<0.01, p<0.001, p<0.0001, respectively. Data are presented as Mean+/−SEM

FIG. 4—Safety of Compounds as Non-Oncogenic

The safety of the compounds were tested in two different human cancer cell lines purchased from ATCC. FIG. 4A cell line PC-3 was of prostate/adenocarcinoma from a Caucasian male, aged 62 years and FIG. 4B cell line PANC-1 was from a pancreatic duct epitheloid carcinoma from a Caucasian male, aged 56 years. Each of the cell lines were seeded in 384 well plates at low density in their growth medium. The day after, the growth medium was removed and replaced by serum free medium. For treatment, compounds (at 3 μM final concentration) were directly added to the cell cultures 16 hours after initial plating. Cultures were then grown for 96 hours. Cells were stained Hoeschst 33342 to visualize and count cell nuclei. Custom module analysis based on Cell Scoring of the MetaXpress software was used for quantification. For each condition, the total number of cells was determined to evaluate the cell amplification. These results indicate the safety of the compounds as being non-oncogenic.

*, **, ***, **** indicates difference from the control, One-way ANOVA, with p<0.05, p<0.01, p<0.001, p<0.0001, respectively. Data are presented as Mean+/−SEM.

FIG. 5—In Vivo Muscle Regeneration: Measurement of Early Phase Expansion and Differentiation of Muscle Stem Cells

Early phase of expansion and subsequent phase of myogenic differentiation of Muscle Stem Cells were evaluated.

FIG. 5A shows the number of Pax7+ cells (fold change) with the control (water) compared to after addition of cirsimaritin or xanthomicrol, respectively. Data are expressed as number of cells per area of injured muscle.

FIG. 5B shows the number of myogenin+ cells (fold change) with the control (water) compared to after addition of cirsimaritin or xanthomicrol, respectively. Data are expressed as number of cells per area of injured muscle.

FIG. 5C shows the size of newly formed myofibres with the control (water) compared to after addition of cirsimaritin or xanthomicrol, respectively. The size of each newly formed myofiber has been measured based on the expression of the eMHC and laminin that allow to recognize and delineate these nascent myofibers. Results are shown as mean myofiber cross-sectional area (μm²).

*, **, ***, **** indicates difference from the control, One-way ANOVA, with p<0.05, p<0.01, p<0.001, p<0.0001, respectively. Data are presented as Mean+/−SEM.

### DETAILED DESCRIPTION OF THE INVENTION

Various preferred features and embodiments of the present invention will now be described by way of non-limiting examples.

Compounds of the Invention

Compounds of the present invention are polymethoxyflavones.

In one embodiment, the present invention relates to a compound of general formula (I):

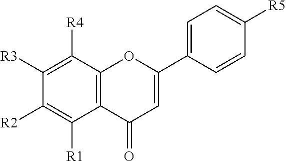

wherein R1, R2, R3, and R4, are each independently H; OH; OMe; O-glycoside; C-glycoside; acylated O-glycoside; acylated C-glycoside; sulfated O-glycoside; sulfated C-glycoside; a halogen; a ketone; an aldehyde; a carboxylic acid; an ester; a primary, secondary, or tertiary amine; a primary or secondary amide; a cyano; a nitro; a sulfonate; a sulfate; an optionally substituted and/or optionally branched C1 to C20 alkyl; an optionally substituted and/or optionally branched, C2 to C20 alkenyl; an optionally substituted and/or optionally branched, C4 to C20 polyalkenyl; an optionally substituted and/or optionally branched C2 to C20 alkynyl, or an optionally substituted and/or optionally branched C4 to C20 polyalkynyl and R5 is H; OH; O-glycoside; C-glycoside; acylated O-glycoside; acylated C-glycoside; sulfated O-glycoside; sulfated C-glycoside; a halogen; a ketone; an aldehyde; a carboxylic acid; an ester; a primary, secondary, or tertiary amine; a primary or secondary amide; a cyano; a nitro; a sulfonate; a sulfate; an optionally substituted and/or optionally branched C1 to C20 alkyl; an optionally substituted and/or optionally branched, C2 to C20 alkenyl; an optionally substituted and/or optionally branched, C4 to C20 polyalkenyl; an optionally substituted and/or optionally branched C2 to C20 alkynyl, or an optionally substituted and/or optionally branched C4 to C20 polyalkynyl.

In some embodiments, a OMe group can cyclize with a neighboring OH group to form a methylene dioxy bridge.

In another embodiment, the invention relates to compounds of general formula (II):

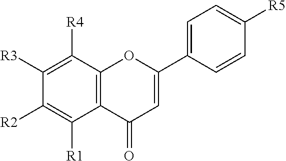

wherein R1, R2, R3, and R4 are each independently H; OH; OMe; O-glycoside; a sulfate; an optionally substituted and/or optionally branched C1 to C20 alkyl; an optionally substituted and/or optionally branched, C2 to C20 alkenyl; or an optionally substituted and/or optionally branched, C4 to C20 polyalkenyl and R5 is H; OH; O-glycoside; a sulfate; an optionally substituted and/or optionally branched C1 to C20 alkyl; an optionally substituted and/or optionally branched, C2 to C20 alkenyl; or an optionally substituted and/or optionally branched, C4 to C20 polyalkenyl.

In some embodiments, a OMe group can cyclize with a neighboring OH group to form a methylene dioxy bridge.

In another embodiment, the invention relates to compounds of general formula (III):

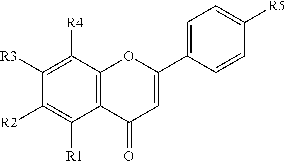

wherein R1, R2, R3, and R4 are each independently H; OH; OMe; O-glycoside; or a sulfate and R5 is H; OH; O-glycoside; or a sulfate.

In some embodiments, a OMe group can cyclize with a neighboring OH group to form a methylene dioxy bridge.

In one embodiment, said compound is Xanthomicrol (5,4′-Dihydroxy-6,7,8-trimethoxyflavone, CAS number 16545-23-6):

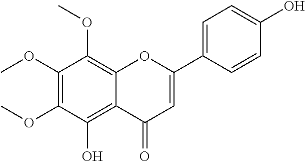 [SMILES: COc1c(OC)c(O)c2c(=O)cc(-c3ccc(O)cc3)oc2c1OC]

Xanthomicrol is also known as 4H-1-Benzopyran-4-one, 5-hydroxy-2-(4-hydroxyphenyl)-6,7,8-trimethoxy; Flavone 4′,5-dihydroxy-6,7,8-trimethoxy; 5-Hydroxy-2-(4-hydroxyphenyl)-6,7,8-trimethoxy-4H-1-benzopyran-4-one; and NSC 79323 with molecular formula C₁₈H₁₁O₇ and molecular weight 344.32.

In one embodiment, said compound is Cirsimaritin (5,4′-Dihydroxy-6,7-dimethoxyflavone, CAS number 6601-62-3):

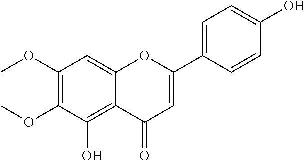 [SMILES: COc1cc2oc(-c3ccc(O)cc3)cc(=O)c2c(O)c1OC]

Cirsimaritin is also known as 4H-1-Benzopyran-4-one, 5-hydroxy-2-(4-hydroxyphenyl)-6,7-dimethoxy; Flavone 4′,5-dihydroxy-6,7-dimethoxy; 5-Hydroxy-2-(4-hydroxyphenyl)-6,7-dimethoxy-4H-1-benzopyran-4-one; 4′,5-Dihydroxy-6,7-dimethoxyflavone; 5,4′-Dihydroxy-6,7-dimethoxyflavone; 6,7-Dimethoxyscutellarein; 6-Methoxyapigenin 7-methyl ether; 6-Methoxygenkwanin; 7-Methylcapillarisin; Cismaritin; Cirsitakaogenin; Cirsumaritin; Scrophulein; Scutellarein 6,7-dimethyl ether; and Skrofulein with molecular formula C₁₇H₁₄O₆ and molecular weight 314.29.

In one embodiment, said compound is Cirsimarin (5,4′-Dihydroxy-6,7-dimethoxyflavone 4′-O-β-D-glucoside, CAS number 13020-19-4):

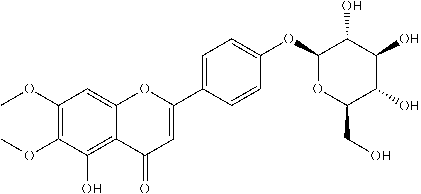 [SMILES: COc1cc2oc(-c3ccc(O[C@@H]4O[C@H](CO)[C@@H](O)[C@H](O)[C@H]4O)cc3)cc(=O)c2c(O)c1OC]

Cirsimarin is also known as 4H-1-Benzopyran-4-one, 2-[4-(β-D-glucopyranosyloxy)phenyl]-5-hydroxy-6,7-dimethoxy; 2-[4-(β-D-Glucopyranosyloxy)phenyl]-5-hydroxy-6,7-dimethoxy-4H-1-benzopyran-4-one; 5,4′-Dihydroxy-6,7-dimethoxyflavone 4′-O-β-D-glucoside; Cirsimaretin; Cirsimarine; Cirsimaritin 4′-glucoside; Cirsimartin; Cirsitakaoside; and Cismaritin with molecular formula C₂₃H₂₄O₁₁ and molecular weight 476.43.

In one embodiment, the invention relates to compounds of general formula (IV)

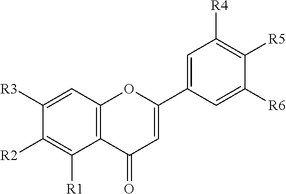

wherein R1, R2, and R3 are each independently H; OH; OMe; O-glycoside; C-glycoside; acylated O-glycoside; acylated C-glycoside; sulfated O-glycoside; sulfated C-glycoside; a halogen; a ketone; an aldehyde; a carboxylic acid; an ester; a primary, secondary, or tertiary amine; a primary or secondary amide; a cyano; a nitro; a sulfonate; a sulfate; an optionally substituted and/or optionally branched C1 to C20 alkyl; an optionally substituted and/or optionally branched, C2 to C20 alkenyl; an optionally substituted and/or optionally branched, C4 to C20 polyalkenyl; an optionally substituted and/or optionally branched C2 to C20 alkynyl, or an optionally substituted and/or optionally branched C4 to C20 polyalkynyl and R4, R5 and R6, are each independently OH; OMe; O-glycoside; C-glycoside; acylated O-glycoside; acylated C-glycoside; sulfated O-glycoside; sulfated C-glycoside; a halogen; a ketone; an aldehyde; a carboxylic acid; an ester; a primary, secondary, or tertiary amine; a primary or secondary amide; a cyano; a nitro; a sulfonate; a sulfate; an optionally substituted and/or optionally branched C1 to C20 alkyl; an optionally substituted and/or optionally branched, C2 to C20 alkenyl; an optionally substituted and/or optionally branched, C4 to C20 polyalkenyl; an optionally substituted and/or optionally branched C2 to C20 alkynyl, or an optionally substituted and/or optionally branched C4 to C20 polyalkynyl.

In some embodiments, a OMe group can cyclize with a neighboring OH group to form a methylene dioxy bridge.

In one embodiment, the invention relates to compounds of general formula (V):

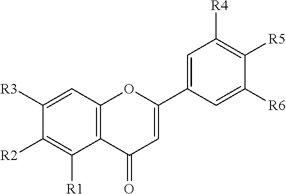

wherein R1, R2 and R3 are each independently H; OH; OMe; O-glycoside; a sulfate; an optionally substituted and/or optionally branched C1 to C20 alkyl; an optionally substituted and/or optionally branched, C2 to C20 alkenyl; or an optionally substituted and/or optionally branched, C4 to C20 polyalkenyl and R4, R5 and R6, are each independently OH; OMe; O-glycoside; a sulfate; an optionally substituted and/or optionally branched C1 to C20 alkyl; an optionally substituted and/or optionally branched, C2 to C20 alkenyl; or an optionally substituted and/or optionally branched, C4 to C20 polyalkenyl.

In some embodiments, a OMe group can cyclize with a neighboring OH group to form a methylene dioxy bridge.

In one embodiment, the invention relates to compounds of general formula (VI):

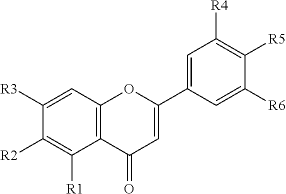

wherein R1, R2 and R3 are each independently H; OH; OMe; O-glycoside; a sulfate and R4, R5 and R6, are each independently OH; OMe; O-glycoside; a sulfate.

In some embodiments, a OMe group can cyclize with a neighboring OH group to form a methylene dioxy bridge.

In one embodiment, said compound is 5,6,7,3′,4′,5′-Hexamethoxyflavone, CAS number 29043-07-0):

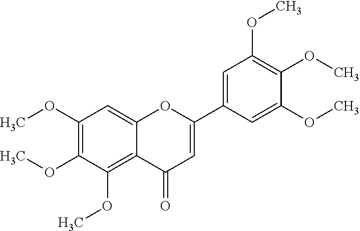

5,6,7,3′,4′,5′-Hexamethoxyflavone is also known as 4H-1-Benzopyran-4-one, 5,6,7-trimethoxy-2-(3,4,5-trimethoxyphenyl); Flavone 3′,4′,5,5′,6,7-hexamethoxy; 5,6,7-Trimethoxy-2-(3,4,5-trimethoxyphenyl)-4H-1-benzopyran-4-one; and 3′,4′,5′,5,6,7-Hexamethoxyflavone with molecular formula C₂₁H₂₂O₈ and molecular weight 402.39.

In one embodiment, the invention relates to compounds of general formula (VII):

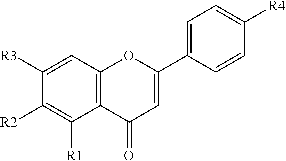

wherein R1 and R2 are each independently H or OH and R3 and R4, are each independently H; OH; OMe; O-glycoside; C-glycoside; acylated O-glycoside; acylated C-glycoside; sulfated O-glycoside; sulfated C-glycoside; a halogen; a ketone; an aldehyde; a carboxylic acid; an ester; a primary, secondary, or tertiary amine; a primary or secondary amide; a cyano; a nitro; a sulfonate; a sulfate; an optionally substituted and/or optionally branched C1 to C20 alkyl; an optionally substituted and/or optionally branched, C2 to C20 alkenyl; an optionally substituted and/or optionally branched, C4 to C20 polyalkenyl; an optionally substituted and/or optionally branched C2 to C20 alkynyl, or an optionally substituted and/or optionally branched C4 to C20 polyalkynyl.

In one embodiment, the invention relates to compounds of general formula (VIII):

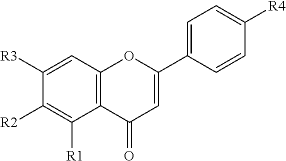

wherein R1 and R2 are each independently H or OH and R3 and R4, are each independently H; OH; OMe; O-glycoside; a sulfate; an optionally substituted and/or optionally branched C1 to C20 alkyl; an optionally substituted and/or optionally branched, C2 to C20 alkenyl; or an optionally substituted and/or optionally branched, C4 to C20 polyalkenyl.

In one embodiment, the invention relates to compounds of general formula (IX):

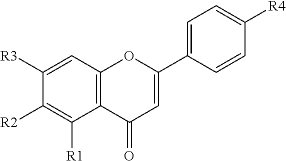

wherein R1 and R2 are each independently H or OH and R3 and R4, are each independently H; OH; OMe; O-glycoside; a sulfate.

In one embodiment, said compound is Ladanein (5,6-Dihydroxy-7,4′-dimethoxyflavone, CAS number 10176-71-3).

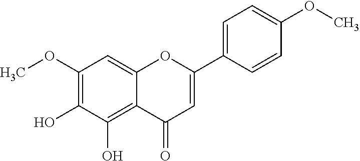

Ladanein is also known as 4H-1-Benzopyran-4-one, 5,6-dihydroxy-7-methoxy-2-(4-methoxyphenyl); Flavone 5,6-dihydroxy-4′,7-dimethoxy; 5,6-Dihydroxy-7-methoxy-2-(4-methoxyphenyl)-4H-1-benzopyran-4-one; 4′,7-Di-O-methylscutellarein; 5,6-Dihydroxy-4′,7-dimethoxyflavone; 5,6-Dihydroxy-7,4′-dimethoxyflavone; BJ 486K; Ladanine; and Scutellarein 4′,7-dimethyl ether with molecular formula C₁₇H₁₄O₆ and molecular weight 314.29.

### Definitions

### General Chemistry Terminology

The term “alkyl” refers to a branched or unbranched saturated hydrocarbon chain having from 1 to 19 carbon atoms, or from 1 to 15 carbon atoms, or from 1 to 9 carbon atoms, or from 1 to 7 carbon atoms, or from 1 to 5 carbon atoms, or from 1 to 3 carbon atoms. This term is exemplified by groups such as methyl, ethyl, n-propyl, iso-propyl, n-butyl, iso-butyl, t-butyl, n-hexyl, n-decyl, tetradecyl, and the like.

The term “substituted alkyl” refers to:

1) an alkyl chain as defined above, having 1, 2, 3, 4 or 5 substituents, (in some embodiments, 1, 2 or 3 substituents) selected from the group consisting of alkyl; alkenyl, alkynyl, alkoxy, cycloalkyl, cycloalkenyl, cycloalkoxy, cycloalkenyloxy, acyl, acylamino, acyloxy, amino, substituted amino, aminocarbonyl, alkoxycarbonylamino, azido, cyano, halogen, hydroxy, keto, thiocarbonyl, carboxy, carboxyalkyl, arylthio, heteroarylthio, heterocyclylthio, thiol, alkylthio, aryl, aryloxy, heteroaryl, aminosulfonyl, aminocarbonylamino, heteroaryloxy, heterocyclyl, heterocyclooxy, hydroxyamino, alkoxyamino, nitro, —S(O)-alkyl, —S(O)-cycloalkyl, —S(O)— heterocyclyl, —S(O)-aryl, —S(O)-heteroaryl, —S(O)2-alkyl, —S(O)2-cycloalkyl, —S(O)2-heterocyclyl, —S(O)2-aryl and —S(O)2-heteroaryl. Unless otherwise constrained by the definition, all substituents may optionally be further substituted by 1, 2 or 3 substituents chosen from alkyl, alkenyl, alkynyl, carboxy, carboxyalkyl, aminocarbonyl, hydroxy, alkoxy, halogen, CF3, amino, substituted amino, cyano, cycloalkyl, heterocyclyl, aryl, heteroaryl, and —S(O)n R<a>, in which R<a> is alkyl, aryl or heteroaryl and n is 0, 1 or 2; or

2) an alkyl chain as defined above that is interrupted by 1-9 atoms (e.g. 1, 2, 3, 4 or 5 atoms) independently chosen from oxygen, sulfur and NR<a>, where R<a> is chosen from hydrogen, alkyl, cycloalkyl, alkenyl, cycloalkenyl, alkynyl, aryl, heteroaryl and heterocyclyl. All substituents may be optionally further substituted by alkyl, alkenyl, alkynyl, carboxy, carboxyalkyl, aminocarbonyl, hydroxy, alkoxy, halogen, CF3, amino, substituted amino, cyano, cycloalkyl, heterocyclyl, aryl, heteroaryl, and —S(O)n R<a>, in which R<a> is alkyl, aryl or heteroaryl and n is 0, 1 or 2; or

3) an alkyl chain as defined above that has both 1, 2, 3, 4 or 5 substituents as defined above and is also interrupted by 1-9 atoms (e.g. 1, 2, 3, 4 or 5 atoms) as defined above.

4) an alkyl chain as defined above in which one of the methylene group is replaced by a carbonyl group to give an oxo group.

5) an alkyl chain as defined above in which one of the methylene group is replaced by a carbonyl group to give an oxo group, and has 1, 2, 3, 4 or 5 substituents as defined above, or is interrupted by 1-9 atoms (e.g. 1, 2, 3, 4 or 5 atoms) as defined above or has both 1, 2, 3, 4 or 5 substituents as defined above and is also interrupted by 1-9 atoms (e.g. 1, 2, 3, 4 or 5 atoms) as defined above.

The term “alkenyl” refers to a type of alkyl chain in which two atoms of the alkyl group form a double bond that is not part of an aromatic group. That is, an alkenyl chain contains the pattern R—C(R)═C(R)—R, wherein R refers to the remaining portions of the alkenyl group, which may be the same or different. Non-limiting examples of an alkenyl chain include —CH═CH2, —C(CH3)═CH2, —CH═CHCH3, —C(CH3)═CHCH3, —CH2-CH═C(CH3)2, and —C(CH3)2-CH═CH2. The alkenyl moiety may be branched, straight chain, or cyclic (in which case, it would also be known as a “cycloalkenyl” group). Alkenyl groups can be optionally substituted.

The alkenyl chain as defined above can be interrupted by 1-8 atoms (e.g. 1, 2, 3, 4 or 5 atoms) independently chosen from oxygen, sulfur and NR<a>, where R<a> is chosen from hydrogen, alkyl, cycloalkyl, alkenyl, cycloalkenyl, alkynyl, aryl, heteroaryl and heterocyclyl. All substituents may be optionally further substituted by alkyl, alkenyl, alkynyl, carboxy, carboxyalkyl, aminocarbonyl, hydroxy, alkoxy, halogen, CF3, amino, substituted amino, cyano, cycloalkyl, heterocyclyl, aryl, heteroaryl, and —S(O)n R<a>, in which R<a> is alkyl, aryl or heteroaryl and n is 0, 1 or 2

The alkenyl chain as defined above can have one of its methylene group replaced by a carbonyl group to give an oxo group.

The alkenyl chain as defined above can have one of its methylene group replaced by an oxo group, and can either have 1, 2, 3, 4 or 5 substituents as defined above, or be interrupted by 1-8 atoms (e.g. 1, 2, 3, 4 or 5 atoms) as defined above, or can have both 1, 2, 3, 4 or 5 substituents as defined above and be also interrupted by 1-8 atoms (e.g. 1, 2, 3, 4 or 5 atoms) as defined above.

The term “alkynyl” refers to a type of alkyl chain in which two atoms of the alkyl group form a triple bond. That is, an alkynyl group contains the pattern R—C≡C—R, wherein R refers to the remaining portions of the alkynyl group, which may be the same or different. Non-limiting examples of an alkynyl group include —C≡CH, —C≡CCH3 and —C≡CCH2CH3. The “R” portion of the alkynyl moiety may be branched, straight chain, or cyclic. Alkynyl groups can be optionally substituted.

The alkynyl chain as defined above can be interrupted by 1-8 atoms (e.g. 1, 2, 3, 4 or 5 atoms) independently chosen from oxygen, sulfur and NR<a>, where R<a> is chosen from hydrogen, alkyl, cycloalkyl, alkenyl, cycloalkenyl, alkynyl, aryl, heteroaryl and heterocyclyl. All substituents may be optionally further substituted by alkyl, alkenyl, alkynyl, carboxy, carboxyalkyl, aminocarbonyl, hydroxy, alkoxy, halogen, CF3, amino, substituted amino, cyano, cycloalkyl, heterocyclyl, aryl, heteroaryl, and —S(O)n R<a>, in which R<a> is alkyl, aryl or heteroaryl and n is 0, 1 or 2 The alkynyl chain as defined above can have one of its methylene group replaced by a carbonyl group to give an oxo group.

The alkynyl chain as defined above can have one of its methylene group replaced by an oxo group, and can either have 1, 2, 3, 4 or 5 substituents as defined above, or be interrupted by 1-8 atoms (e.g. 1, 2, 3, 4 or 5 atoms) as defined above, or can have both 1, 2, 3, 4 or 5 substituents as defined above and be also interrupted by 1-8 atoms (e.g. 1, 2, 3, 4 or 5 atoms) as defined above.

The term “polyalkenyl” refers to a chain in which more than one pair of atoms of the alkyl group form a double bond that is not part of an aromatic group. That is, a polyalkenyl chain contains several R—C(R)═C(R)—R patterns, wherein R refers to the remaining portions of the alkenyl group, which may be the same or different. Non-limiting examples of a polyalkenyl chain include —CH═CH—CH═CH—, —CH2-CH═CCH3-CH2-CH2-CH═C(CH3)2, and —CH2-CH═CCH3-CH2-CH2-CH═CCH3-CH2-CH2-CH═C(CH3)2. The polyalkenyl moiety may be branched, or straight chain. The polyalkenyl moiety containing two double bond may be cyclic (in which case, it would also be known as a “cyclodialkenyl” group). Non limiting example of cyclodialkenyl groups include cyclopentadiene and cyclohexadiene groups. Polyalkenyl groups can be optionally substituted.

The polyalkenyl chain as defined above can be interrupted by 1-7 atoms (e.g. 1, 2, 3, 4 or 5 atoms) independently chosen from oxygen, sulfur and NR<a>, where R<a> is chosen from hydrogen, alkyl, cycloalkyl, alkenyl, cycloalkenyl, alkynyl, aryl, heteroaryl and heterocyclyl.

The polyalkenyl chain as defined above may have one of its methylene group replaced by a carbonyl group to give an oxo group.

The polyalkenyl chain as defined above can have one of its methylene group replaced by an oxo group, and can either have 1, 2, 3, 4 or 5 substituents as defined above, or be interrupted by 1-7 atoms (e.g. 1, 2, 3, 4 or 5 atoms) as defined above, or can have both 1, 2, 3, 4 or 5 substituents as defined above and be also interrupted by 1-7 atoms (e.g. 1, 2, 3, 4 or 5 atoms) as defined above.

The term “polyalkynyl” refers to a chain in which more than one pair of atoms of the alkyl group form a triple bond. That is, a polyalkynyl group contains several patterns R—C≡C—R, wherein R refers to the remaining portions of the alkynyl group, which may be the same or different. Non-limiting example of a polyalkynyl group include —CH2-CH2-C≡C—C≡CH. The “R” portion of the polyalkynyl moiety may be branched, straight chain, or cyclic. Alkynyl groups can be optionally substituted.

The polyalkynyl chain as defined above can be interrupted by 1-7 atoms (e.g. 1, 2, 3, 4 or 5 atoms) independently chosen from oxygen, sulfur and NR<a>, where R<a> is chosen from hydrogen, alkyl, cycloalkyl, alkenyl, cycloalkenyl, alkynyl, aryl, heteroaryl and heterocyclyl.

The polyalkynyl chain as defined above may have one of its methylene group replaced by a carbonyl group to give an oxo group.

The polyalkynyl chain as defined above can have one of its methylene group replaced by an oxo group, and can either have 1, 2, 3, 4 or 5 substituents as defined above, or be interrupted by 1-7 atoms (e.g. 1, 2, 3, 4 or 5 atoms) as defined above, or can have both 1, 2, 3, 4 or 5 substituents as defined above and be also interrupted by 1-7 atoms (e.g. 1, 2, 3, 4 or 5 atoms) as defined above.

As used herein, the term “ring” refers to any covalently closed structure. Rings include, for example, carbocycles (e.g., aryls and cycloalkyls), heterocycles (e.g., heteroaryls and non-aromatic heterocycles), aromatics (e.g. aryls and heteroaryls), and non-aromatics (e.g., cycloalkyls and non-aromatic heterocycles). Rings can be optionally substituted. Rings can form part of a ring system. As used herein, the term “ring system” refers to two or more rings, wherein two or more of the rings are fused. The term “fused” refers to structures in which two or more rings share one or more bonds.

The term “halogen atom” may refer to a fluorine atom, a chlorine atom, a bromine atom or an iodine atom.

The term “glycoside” refers to a compound in which at least one sugar is bound to another functional group via a glycosidic bond. Typically the glycosidic chain can comprise 1 to 4 sugar units.

The term “glycosidic bond” refers to a bond formed between the hemiacetal or hemiketal group of a sugar and the chemical group of a compound. The chemical group can be —OH (O-glycoside), or —CR1R2R3 (C-glycoside).

The terms “acylated O-glycoside” and “acylated C-glycoside” refer to a compound in which at least one hydroxyl of the glycosidic chain is esterified by an organic acid. Typical examples or organic acid may comprise acetic, substituted benzoic, cinnamic (i.e. caffeic, ferulic, p-coumaric), and/or phenylpropanoic (i.e. dihydrocaffeic) acids.

The terms “sulfated O-glycoside” and “sulfated C-glycoside” refer to a compound in which at least one hydroxyl of the glycosidic chain is esterified by sulfuric acid.

The term “methylene dioxy” may refer to functional group with the structural formula R—O—CH2-O—R′, connected to the rest of a molecule by two chemical bonds.

The term “analogue” as used herein is understood to refer to a compound having a structure similar to that of another one, but differing from it in respect of a certain component. A “derivative” is a compound that can be imagined to arise or is actually be synthesized from a parent compound by replacement of one or more atoms with another atom or group of atoms.

In one embodiment of the invention, compounds of the invention modulate muscle stem cell function to maintain or increase skeletal muscle function and/or mass in a subject, and/or substantially prevent or reduce muscle wasting in a subject, and/or to enhance muscle repair after an injury, for example, by accelerating the repair of myofibers or decreasing fibrosis and muscle stiffness or decreasing muscle fat infiltration.

In another embodiment of the invention, compounds of the invention modulate muscle stem cell function by proliferation and/or differentiation of skeletal muscle stem cells.

In a further embodiment of the invention, compounds of the invention modulate muscle stem cell function by myogenesis.

Compositions of the Invention

Compositions comprise one or more compounds of the invention. Compositions of the invention can be, for example, nutritional compositions or pharmaceutical compositions. Nutritional compositions are the preferred embodiment of the invention.

Nutritional Compositions

In one embodiment, the composition is a nutritional composition. The nutritional composition can be any kind of composition that is suitable for human and/or animal consumption. In another embodiment, the nutritional compositions of the invention can comprise plant extracts rich in compounds of the invention or fortified by compounds of the invention.

For example, the composition may be selected from the group consisting of food compositions, dietary supplements, nutritional compositions, nutraceuticals, powdered nutritional products to be reconstituted in water or milk before consumption, food additives, medicaments, beverages and drinks.

In an embodiment, the composition is an oral nutritional supplement (ONS), a complete nutritional formula, a pharmaceutical, a medical or a food product. In a preferred embodiment, the composition is administered to the individual as a beverage. The composition may be stored in a sachet as a powder and then suspended in a liquid such as water for use.

In some instances where oral or enteral administration is not possible or not advised, the composition may also be administered parenterally.

In some embodiments, the composition is administered to the individual in a single dosage form, i.e. all compounds are present in one product to be given to an individual in combination with a meal. In other embodiments, the composition is co-administered in separate dosage forms, with one or more compounds of the invention separate from other components of the composition separately. For example, a food composition comprising a compound of the invention may be administered separately from a beverage composition comprising a compound of the invention.

A “food composition” or “beverage composition” means a product or composition that is intended for ingestion by an individual such as a human or animal and provides at least one compound of the invention to the individual. The compositions of the present disclosure, including the many embodiments described herein, can comprise, consist of, or consist essentially of the essential elements and limitations described herein, as well as any additional or optional ingredients, components, or limitations described herein or otherwise useful in a diet.

The nutritional composition may be considered a “complete nutrition composition” which means that it contains sufficient types and levels of macronutrients (protein, fats and carbohydrates) and micronutrients to be sufficient to be a sole source of nutrition for the individual to which the composition is administered. Individuals can receive 100% of their nutritional requirements from such complete nutritional compositions.

Nutritional compositions may contain compounds of the invention in amount of from 0.01 mg to about 1 g, preferably from 0.1 mg to 1 g, even more preferably from 1 mg to about 1 g per serving.

The effective amount of a composition according to the present invention which is required to achieve a therapeutic effect will vary with the particular composition, the route of administration, the age and condition of the recipient, and the particular disorder or disease being treated. Certain types of these nutritional compositions may be regulated as pharmaceuticals depending on the regulatory laws in a particular country or region.

As an illustration, a RTD (ready to drink) composition contains from 0.01 mg to 500 mg of each active ingredient per serving, more preferably about 250 mg per serving.

In addition to the one or more compounds of the invention, the composition can further comprise a protein source from animal or plant origin, for example milk proteins, soy proteins, and/or pea proteins. In a preferred embodiment, the protein source is selected from the group consisting of whey protein; casein protein; pea protein; soy protein; wheat protein; corn protein; rice protein; proteins from legumes, cereals and grains; and combinations thereof. Additionally or alternatively, the protein source may comprise a protein from nuts and/or seeds.

The protein source may comprise whey protein. The whey protein may be unhydrolyzed or hydrolyzed whey protein. The whey protein may be any whey protein, for example the whey protein can be selected from the group consisting of whey protein concentrates, whey protein isolates, whey protein micelles, whey protein hydrolysates, acid whey, sweet whey, modified sweet whey (sweet whey from which the caseino-glycomacropeptide has been removed), a fraction of whey protein, and any combination thereof. In a preferred embodiment, the whey protein comprises whey protein isolate and/or modified sweet whey.

As noted above, the protein source can be from animal or plant origin, for example milk proteins, soy proteins, and/or pea proteins. In an embodiment, the protein source comprises casein. Casein may be obtained from any mammal but is preferably obtained from cow milk and preferably as micellar casein.

In an embodiment of the invention, the nutritional composition comprises protein in an amount such that the intake of protein, preferably whey, is 5-50 g protein per day, such as from 12-40 g protein per day, preferably from 15-30 g protein per day, such as from 16-25 g protein per day, even more preferably 20 g protein per day.

The nutritional composition can comprise one or more branched chain amino acids. For example, the composition can comprise leucine, isoleucine and/or valine. The protein source in the composition may comprise leucine in free form and/or leucine bound as peptides and/or proteins such as dairy, animal or vegetable proteins. In an embodiment, the composition comprises the leucine in an amount up to 10 wt % of the dry matter of the composition. Leucine can be present as D- or L-leucine and preferably the L-form. If the composition comprises leucine, the composition can be administered in a daily dose that provides 0.01 to 0.04 g of the leucine per kg body weight, preferably 0.02 to 0.035 g of the leucine per kg body weight. Such doses are particularly applicable to complete nutrition compositions, but one of ordinary skill will readily recognize how to adapt these doses for an oral nutritional supplement (ONS).

In an embodiment, the composition comprising one or more compounds of the invention further comprises a fatty acid. The fatty acid may be any fatty acid and may be one or more fatty acids, such as a combination of fatty acids. The fatty acid preferably comprises an essential fatty acid, such as the essential polyunsaturated fatty acids, namely linoleic acid (C18:2n−3) and α-linolenic acid (C18:3n−3). The fatty acid may comprise long-chain polyunsaturated fatty acids, such as eicosapentaenoic acid (C20:5n−3), arachidonic acid (C20:4n−6), docosahexaenoic acid (C22:6n−3), or any combination thereof. In a preferred embodiment, the fatty acid comprises an n−3 (omega 3) fatty acid and/or an n−6 (omega 6) fatty acid. The fatty acid preferably comprises eicosapentaenoic acid.

The fatty acid may be derived from any suitable source containing fatty acids, such as coconut oil, rapeseed oil, soya oils, corn oil, safflower oil, palm oil, sunflower oil or egg yolk. The source of the fatty acid is preferably fish oil.

The n−3 fatty acid according to the present invention is usually at least 10 wt %, preferably at least 15 wt %, based on total lipid content. In a preferred embodiment the daily amount is from 500 mg to 2.5 g, preferably 1 g to 1.5 g n−3 fatty acid per day.

Compositions of the invention comprising at least one compound of the invention may additionally comprise anti-inflammatory compounds or antioxidant compounds. For example, the additional antioxidants may be provided as food compositions that are rich in antioxidants or as extracts thereof. A food composition that is “rich in antioxidants” has an ORAC (oxygen radical absorbance capacity) rating of at least 100 per 100 g of the composition.

In one embodiment, the composition comprises a compound of the invention and at least a protein source, an amino acid and an n−3 fatty acid.

In another embodiment, the composition comprises a compound of the invention and further comprises an anti-oxidant compound.

In an embodiment, the composition further comprises a compound of the invention and at least one vitamin such as vitamin D or B-complex vitamins. Compositions according to the invention may, for example, comprise Vitamin D in an amount of from 800 to 1200 IU per serving.

The nutritional composition of the invention can be administered to an individual such as a human, e.g., an elderly human, in a therapeutically effective dose. The therapeutically effective dose can be determined by the person skilled in the art and will depend on a number of factors known to those of skill in the art, such as the severity of the condition and the weight and general state of the individual.

In one embodiment of the invention, the nutrition composition is administered to a subject in combination with a regime of exercise or physical activity.

The nutritional composition of the invention can be formulated to be administered to an animal, in the form of animal treats (e.g., biscuits), or dietary supplements. The compositions may be a dry composition (e.g., kibble), semi-moist composition, wet composition, or any mixture thereof. In another embodiment, the nutritional composition is a dietary supplement such as a gravy, drinking water, beverage, yogurt, powder, granule, paste, suspension, chew, morsel, treat, snack, pellet, pill, capsule, tablet, or any other suitable delivery form.

The moisture content can vary depending on the nature of the composition. In a one embodiment, the composition can be a complete and nutritionally balanced pet food. In this embodiment, the pet food may be a “wet food”, “dry food”, or food of intermediate moisture content. “Wet food” describes pet food that is typically sold in cans or foil bags, and has a moisture content typically in the range of about 70% to about 90%. “Dry food” describes pet food which is of a similar composition to wet food, but contains a limited moisture content, typically in the range of about 5% to about 15% or 20%, and therefore is presented, for example, as small biscuit-like kibbles. In one embodiment, the compositions have moisture content from about 5% to about 20%. Dry food products include a variety of foods of various moisture contents, such that they are relatively shelf-stable and resistant to microbial or fungal deterioration or contamination. Also included are dry food compositions which are extruded food products, such as pet foods, or snack foods for either humans or companion animals.

The nutritional composition may be administered to an individual in an amount sufficient to prevent or at least partially reduce the risk of developing a disease or condition sarcopenia in instances where the condition of sarcopenia has yet not been developed in the individual. Such an amount is defined to be “a prophylactically effective dose.” Again, the precise amounts depend on a number of factors relating to the individual, such as their weight, health and how much muscle functionality (e.g. muscle strength, gait speed, etc.) is being lost.

The nutritional composition is preferably administered as a supplement to the diet of an individual daily or at least twice a week. In an embodiment, the composition is administered to the individual consecutively for a number of days, preferably until an increase in muscle functionality (e.g. muscle strength, gait speed, etc.) relative to that before administration is achieved. For example, the composition can be administered to the individual daily for at least 30, 60 or 90 consecutive days. As another example, the composition can be administered to the individual for a longer period, such as a period of 1, 2, 3, 4, 5, 6, 7, 8, 9 or 10 years.

In a preferred embodiment, the nutritional composition is administered to the individual for at least 3 months, for example a period of 3 months to 1 year, and preferably for at least 6 months.

The above examples of administration do not require continuous daily administration with no interruptions. Instead, there may be some short breaks in the administration, such as a break of two to four days during the period of administration. The ideal duration of the administration of the composition can be determined by those of skill in the art.

In a preferred embodiment, the composition is administered to the individual orally or enterally (e.g. tube feeding). For example, the composition can be administered to the individual as a beverage, a capsule, a tablet, a powder or a suspension.

Pharmaceutical Compositions

Compositions of the invention comprise at least one compound of the invention which may be formulated as pharmaceutical compositions or nutritional compositions which are regulated as pharmaceutical compositions.

Pharmaceutical compositions, contain, for example, from about 10% to about 100%, preferably from about 20% to about 60%, of the active compounds of the invention. Pharmaceutical preparations for enteral or parenteral administration are, for example, those in unit dosage forms, such as sugar-coated tablets, tablets, capsules or suppositories, and furthermore ampoules. If not indicated otherwise, these are prepared in a manner known per se, for example by means of conventional mixing, granulating, sugar-coating, dissolving or lyophilizing processes. It will be appreciated that the unit content contained in an individual dose of each dosage form need not in itself constitute an effective amount since the necessary effective amount can be reached by administration of a plurality of dosage units.

In particular, a therapeutically effective amount of a compound of the invention may be administered simultaneously or sequentially and in any order, and for combinations, the components may be administered separately or as a fixed combination. For example, when used in a method of treatment of cachexia associated with chemotherapy for cancer according to the present invention may comprise (i) administration of the combination partner (a) in free or pharmaceutically acceptable salt form and (ii) administration of a combination partner (b) in free or pharmaceutically acceptable salt form, simultaneously or sequentially in any order, in jointly therapeutically effective amounts, preferably in synergistically effective amounts, e.g. in daily dosages corresponding to the amounts described herein.

The individual combination partners of the invention can be administered separately at different times during the course of therapy or concurrently in divided or single combination forms. The invention is embracing all such regimes of simultaneous or alternating treatment and the term “administering” is to be interpreted accordingly.

The effective dosage may vary depending on the particular compound or pharmaceutical composition employed, the mode of administration, the condition being treated, the severity of the condition being treated. Thus, the dosage regimen is selected in accordance with a variety of factors including the route of administration and the renal and hepatic function of the patient. A physician, clinician or veterinarian of ordinary skill can readily determine and prescribe the effective amount of the single active ingredients required to prevent, counter or arrest the progress of the condition. Optimal precision in achieving concentration of the active ingredients within the range that yields efficacy without toxicity requires a regimen based on the kinetics of the active ingredients' availability to target sites.

The compounds of formulae (I) to (IX) can be administered by any route including orally, parenterally, e.g., intraperitoneal, intravenously, intramuscularly, subcutaneously, intratumorally, or rectally, or enterally. Preferably the compounds of formulae (I) to (IX) are administered orally, preferably at a daily dosage of 1-300 mg/kg body weight or, for most larger primates, a daily dosage of 50-5000, preferably 500-3000 mg. A preferred oral daily dosage is 1-75 mg/kg body weight or, for most larger primates, a daily dosage of 10-2000 mg, administered as a single dose or divided into multiple doses, such as twice daily dosing.

The compound of formulae (I) to (IX), is preferably administered orally to a human in a dosage in the range of about 100 to 2000 mg/day, more preferably 500 to 1500 mg/day, e.g. 1000 mg/day and most preferably 750 mg/day to 1500 mg/day.

In one embodiment of the invention, the composition of the invention is provided for use to maintain or increase muscle function and/or mass in a subject, and/or substantially prevent or reduce muscle wasting in a subject.

In another embodiment of the invention, the composition is a nutritional composition is provided to maintain or increase muscle function and/or mass in a subject, and/or substantially prevent or reduce muscle wasting in a subject.

In another embodiment of the invention, the composition is a nutritional composition comprising a compound of the invention wherein modulation of muscle stem cell function is measured by increase in the number of muscle stem cells and/or myoblasts and/or myotubes.

In one embodiment of the invention, the nutritional composition is provided to maintain or increase muscle function and/or mass in a subject, and/or substantially prevent or reduce muscle wasting in a subject.

In another embodiment of the invention, the nutritional composition is provided to prevent or treat cachexia or precachexia; sarcopenia, myopathy, dystrophy, and/or recovery after muscle injury or surgery.

In a further embodiment of the invention, the nutritional composition of the invention is provided to be used to prevent or treat cachexia wherein cachexia is associated with a disease selected from cancer, chronic heart failure, renal failure, chronic obstructive pulmonary disease, AIDS, autoimmune disorders, chronic inflammatory disorders, cirrhosis of the liver, anorexia, chronic pancreatitis, metabolic acidosis and/or neurodegenerative disease.

In a preferred embodiment of the invention, the nutritional composition of the invention is provided to be used to prevent or treat cachexia or precachexia associated with cancer.

In another preferred embodiment of the invention, the nutritional composition of the invention is provided to be used in the treatment of cachexia associated with cancer is selected from pancreas cancer, esophagus, stomach, bowel, lung and/or liver cancer.

In another embodiment of the invention, the composition is a pharmaceutical composition of the invention provided to maintain or increase muscle function and/or mass in a subject, and/or substantially prevent or reduce muscle wasting in a subject.

In another embodiment of the invention, the composition is a pharmaceutical composition comprising a compound of the invention wherein modulation of muscle stem cell function is measured by increase in the number of muscle stem cells and/or myoblasts and/or myotubes.

In one embodiment of the invention, the pharmaceutical composition is provided to maintain or increase muscle function and/or mass in a subject, and/or substantially prevent or reduce muscle wasting in a subject.

In another embodiment of the invention, the pharmaceutical composition is provided to prevent or treat cachexia or precachexia; sarcopenia, myopathy, dystrophy, and/or recovery after muscle injury or surgery.

In a further embodiment of the invention, the pharmaceutical composition of the invention is provided to be used to prevent or treat cachexia wherein cachexia is associated with a disease selected from cancer, chronic heart failure, renal failure, chronic obstructive pulmonary disease, AIDS, autoimmune disorders, chronic inflammatory disorders, cirrhosis of the liver, anorexia, chronic pancreatitis, metabolic acidosis and/or neurodegenerative disease.

In a preferred embodiment of the invention, the pharmaceutical composition of the invention is provided to be used to prevent or treat cachexia or precachexia associated with cancer.

In another preferred embodiment of the invention, the pharmaceutical composition of the invention is provided to be used in the treatment of cachexia associated with cancer is selected from pancreas cancer, esophagus, stomach, bowel, lung and/or liver cancer.

In a further embodiment of the invention, the compound or composition of the invention is provided to be used in the manufacture of a medicament for the prevention and/or treatment of cachexia.

Combination Together with Chemotherapy Agents to Treat Cancer

A combination of the invention includes at least one compound of the invention and a chemotherapy agent to treat cancer. In one embodiment, a nutritional composition of the invention is administered with a chemotherapy agent to treat cancer together or separately administered.

Administration of a combination results in a surprising beneficial effect of slowing down, arresting or reversing the progress of muscle wasting, e.g. less cachexia, an improved quality of life and a decreased mortality and morbidity, compared to a monotherapy applying only one of the pharmaceutically active ingredients.

In one embodiment of the invention, the nutritional composition of the invention may be administered in combination with a therapeutic anti-cancer compound.

In another embodiment of the invention, the nutritional composition of the invention may be administered separately or sequentially before or after administration of a therapeutic anti-cancer compound.

Kit of Parts

A combination preparation can be defined as a “kit of parts” in the sense that it can be dosed independently or by use of different fixed combinations with distinguished amounts of combination i.e. simultaneously or at different time points. The parts of the kit of parts can then, e.g., be administered simultaneously or chronologically staggered, that is at different time points and with equal or different time intervals for any part of the kit of parts. Very preferably, the time intervals are chosen such that the effect on the treated disease in the combined use of the parts is larger than the effect which would be obtained by use of only any one of the combination partners (a) and (b). The ratio of the total amounts of the combination partner (a) to the combination partner (b) to be administered in the combined preparation can be varied, e.g. in order to cope with the needs of a patient sub-population to be treated or the needs of the single patient which different needs can be due to the particular disease, age, sex, body weight, etc. of the patients. Preferably, there is at least one beneficial effect, e.g., a mutual enhancing of the effect of the combination partners.

In one embodiment of the invention, a kit of parts is provided for the prevention or treatment of cachexia or precachexia comprising a compound or composition of the invention.

In another embodiment of the invention, a kit of parts is provided for the prevention or treatment of cachexia or precachexia comprising a compound of the invention to be administered separately or together with an anti-cancer treatment.

In another embodiment of the invention, a kit of parts is provided for maintaining or increasing muscle function and/or muscle mass in a subject and/or substantially preventing or reducing muscle wasting in a subject with sarcopenia, myopathy, dystrophy and/or recover after muscle injury or surgery comprising a compound or a composition of the invention.

In a further embodiment of the invention, a kit of parts is provided wherein the kit additionally comprises instructions for dietary intervention of high caloric, high protein, high carbohydrate, Vitamin B3, B12 and/or Vitamin D supplementation, antioxidants, omega fatty acids and/or polyphenols for daily administration.

Combination of Compounds and Compositions of the Invention with Dietary Intervention

The term “dietary intervention” refers to an external factor applied to a subject which causes a change in the subject's diet. In one embodiment, the dietary intervention is a high calorie diet. In another embodiment, the dietary intervention is a high protein and/or carbohydrate diet. In another embodiment, the dietary intervention is a diet supplemented with vitamins and minerals, in particular vitamin B12 and/or vitamin D. In another embodiment the dietary intervention is supplemented with anti-oxidants, for example N-acetyl-cysteine. In a further embodiment, the dietary intervention is supplemented with omega fatty acids. In a further embodiment, the dietary intervention is supplemented with a polyphenol or a vitamin B3 that increases mitochondrial activity, for example nicotinamide riboside.

The diet may be one which is adjusted to the starting body weight of the subject.

The dietary intervention may comprise administration of at least one diet product. The diet product may be a meal replacement product or a supplement product which may, for example, increase the subject's appetite. The diet product may include food products, drinks, pet food products, food supplements, nutraceuticals, food additives or nutritional formulae. Example oral nutritional supplements include Nestlé Boost, Resource and Meritene products.

In one embodiment of the invention, a compound or a composition of the invention may be used in a method of prevention or treatment of cachexia in combination with a dietary intervention of high caloric, high protein, high carbohydrate, Vitamin B3, Vitamin B12 and/or Vitamin D supplementation, antioxidants, omega fatty acids and/or polyphenols.

Cachexia and Related Diseases

The invention provides compounds, compositions and methods of preventing and/or treating cachexia or skeletal muscle wasting syndrome by modulating skeletal muscle stem cells. Cachexia is a complex metabolic syndrome associated with underlying illness and characterized by loss of muscle with or without loss of fat mass. The prominent clinical feature of cachexia is weight loss in adults (corrected for fluid retention) or growth failure in children (excluding endocrine disorders).

Cachexia is often seen in patients with diseases such as cancer, chronic heart failure, renal failure, chronic obstructive pulmonary disease, AIDS, autoimmune disorders, chronic inflammatory disorders, cirrhosis of the liver, anorexia, chronic pancreatitis and/or metabolic acidosis and neurodegenerative disease.

There are certain types of cancer wherein cachexia is particularly prevalent, for example, pancreas, esophagus, stomach, bowel, lung and/or liver cancer.

The internationally recognised diagnostic criterion for cachexia is weight loss greater than 5% over a restricted time, for example 6 months, or weight loss greater than 2% in individuals already showing depletion according to current body weight and height (body-mass index [BMI]<20 kg/m²) or skeletal muscle mass (measured by DXA, MRI, CT or bioimpedance). Cachexia can develop progressively through various stages—precachexia to cachexia to refractory cachexia. Severity can be classified according to degree of depletion of energy stores and body protein (BMI) in combination with degree of ongoing weight loss.

In particular, cancer cachexia has been defined as weight loss >5% over past 6 months (in absence of simple starvation); or BMI <20 and any degree of weight loss >2%; or appendicular lean mass consistent with low muscle mass (males <7·26 kg/m²; females <5·45 kg/m²) and any degree of weight loss >2% (Fearon et al. 2011).

Precachexia may be defined as weight loss 55% together with anorexia and metabolic change. At present there are no robust biomarkers to identify those precachectic patients who are likely to progress further or the rate at which they will do so. Refractory cachexia is defined essentially on the basis of the patient's clinical characteristics and circumstances.

It may be appreciated that the compounds, compositions and methods of the present invention may be beneficial for the prevention and/or treatment of the condition of precachexia as well as cachexia in particular to maintain or improve skeletal muscle mass and/or muscle function.

In one embodiment of the invention, the invention provides a method of treatment of cachexia or precachexia comprising administering to a human or animal subject an effective amount of a compound of the invention.

In another embodiment of the invention, the invention provides a method of treatment of cachexia or precachexia comprising administering to a human or animal subject an effective amount of a compound of the invention wherein cachexia or precachexia is associated with a disease selected from cancer, chronic heart failure, renal failure, chronic obstructive pulmonary disease, AIDS, autoimmune disorders, chronic inflammatory disorders, cirrhosis of the liver, anorexia, chronic pancreatitis, metabolic acidosis and/or neurodegenerative disease.

In a preferred embodiment of the invention, the invention provides a method of treatment of cancer cachexia is associated with cancer is selected from pancreas, esophagus, stomach, bowel, lung and/or liver cancer.

In yet another embodiment of the invention, the invention provides a method of treatment wherein treatment of cancer cachexia is measured by reducing body weight loss, preventing body weight loss, maintaining body weight or increasing body weight.

In another embodiment of the invention, a compound or a composition of the invention may be used in a method of treatment wherein cancer cachexia is a result of treatment for cancer with a chemotherapeutic agent.

In a further embodiment of the invention, a compound or a composition of the invention may be used in a method of prevention or treatment of cachexia in combination with a dietary intervention of high caloric, high protein, high carbohydrate, Vitamin B3, Vitamin B12 and/or Vitamin D supplementation, antioxidants, omega fatty acids, and/or polyphenols.

Sarcopenia and Related Diseases

Sarcopenia can be characterized by one or more of low muscle mass, low muscle strength and low physical performance.

Sarcopenia can be diagnosed in a subject based on the definition of the AWGSOP (Asian Working Group for Sarcopenia in Older People), for example as described in Chen et al., 2014. Low muscle mass can generally be based on low appendicular lean mass normalized to height square (ALM index), particularly ALM index less than 7.00 kg/m2 for men and 5.40 kg/m2 for women. Low physical performance can generally be based on gait speed, particularly gait speed of <0.8 m/sec. Low muscle strength can generally be based on low hand grip strength, particularly hand grip strength less than 26 kg in men and less than 18 kg in women.

Sarcopenia can be diagnosed in a subject based on the definition of the EWGSOP (European Working Group for Sarcopenia in Older People), for example as described in Crutz-Jentoft et al., 2010. Low muscle mass can generally be based on low appendicular lean mass normalized to height square (ALM index), particularly ALM index less than 7.23 kg/m2 for men and 5.67 kg/m2 for women. Low physical performance can generally be based on gait speed, particularly gait speed of <0.8 m/sec. Low muscle strength can generally be based on low hand grip strength, particularly hand grip strength less than 30 kg in men and less than 20 kg in women.

Sarcopenia can be diagnosed in a subject based on the definition of the Foundation for the National Institutes of Health (FNIH), for example as described in Studenski et al., 2014. Low muscle mass can generally be based on low appendicular lean mass (ALM) normalized to body mass index (BMI; kg/m2), particularly ALM to BMI less than 0.789 for men and 0.512 for women. Low physical performance can generally be based on gait speed, particularly gait speed of <0.8 m/sec. Low muscle strength can generally be based on low hand grip strength, particularly hand grip strength less than 26 kg in men and less than 16 kg in women. Low muscle strength can also generally be based on low hand grip strength to body mass index, particularly hand grip strength to body mass index less than 1.00 in men and less than 0.56 in women.

The D3-creatine dilution method is another approach to measure muscle mass. This method is becoming more widely accepted as a robust standard and potentially a future alternative to DXA. The D3-creatine dilution method has been described previously in Clark et al. (1985) and Stimpson et al. (2013).

It may be appreciated that the compounds, compositions and methods of the present invention may be beneficial to prevent and/or treat sarcopenia and/or related conditions, in particular, to maintain or improve skeletal muscle mass and/or muscle function.

Myopathy and Related Conditions

Myopathies are neuromuscular disorders in which the primary symptom is muscle weakness due to dysfunction of muscle fiber. Other symptoms of myopathy can include muscle cramps, stiffness, and spasm. Myopathies can be inherited (such as the muscular dystrophies) or acquired (such as common muscle cramps).

Myopathies are grouped as follows: (i) congenital myopathies: characterized by developmental delays in motor skills; skeletal and facial abnormalities are occasionally evident at birth (ii) muscular dystrophies: characterized by progressive weakness in voluntary muscles; sometimes evident at birth (iii) mitochondrial myopathies: caused by genetic abnormalities in mitochondria, cellular structures that control energy; include Kearns-Sayre syndrome, MELAS and MERRF glycogen storage diseases of muscle: caused by mutations in genes controlling enzymes that metabolize glycogen and glucose (blood sugar); include Pompe's, Andersen's and Cori's diseases (iv) myoglobinurias: caused by disorders in the metabolism of a fuel (myoglobin) necessary for muscle work; include McArdle, Tarui, and DiMauro diseases (v) dermatomyositis: an inflammatory myopathy of skin and muscle (vi) myositis ossificans: characterized by bone growing in muscle tissue (vii) familial periodic paralysis: characterized by episodes of weakness in the arms and legs (viii)polymyositis, inclusion body myositis, and related myopathies: inflammatory myopathies of skeletal muscle (ix) neuromyotonia: characterized by alternating episodes of twitching and stiffness; and stiff-man syndrome: characterized by episodes of rigidity and reflex spasms common muscle cramps and stiffness, and (x) tetany: characterized by prolonged spasms of the arms and legs. (Reference: https://www.ninds.nih.gov/disorders/all-disorders/myopathy-information-page).

It may be appreciated that the compounds, compositions and methods of the present invention may be beneficial to prevent and/or treat the aforementioned diseases or conditions, in particular, to maintain or improve skeletal muscle mass and/or muscle function.

Muscular Dystrophy

Muscular dystrophy are a group of genetic diseases characterized by progressive weakness and degeneration of the skeletal or voluntary muscles which control movement. Major types of muscular dystrophy include: Duchenne muscular dystrophy, Becker muscular dystrophy, limb-girdle muscular dystrophy, facioscapulohumeral muscular dystrophy, congenital muscular dystrophy, oculopharyngeal muscular dystrophy, distal muscular dystrophy, Emery-Dreifuss muscular dystrophy and myotonic dystrophy.

(Reference: https://www.medicalnewstoday.com/articles/187618.php)

It may be appreciated that the compounds, compositions and methods of the present invention may be beneficial to prevent and/or treat the aforementioned diseases or conditions, in particular, to maintain or improve skeletal muscle mass and/or muscle function,

Recovery after Muscle Injury from Surgery and Muscle Traumas

Muscle injuries can be caused by bruising, stretching or laceration causing acute or chronic soft tissue injury that occurs to a muscle, tendon, or both. It may occur as a result of fatigue, overuse, or improper use of a muscle. It may occur after physical trauma such as a fall, fracture or overuse during physical activity. Muscle injuries may also occur after surgery such as joint replacement arthroscopic surgery.

It may be appreciated that the compounds, compositions and methods of the present invention may be beneficial to prevent and/or treat the aforementioned conditions of recovery after surgery and/or muscle trauma, in particular, to maintain or improve skeletal muscle mass and/or muscle function.

Method of Treatment

It is to be appreciated that all references herein to treatment include curative, palliative and prophylactic treatment; although in the context of the invention references to preventing are more commonly associated with prophylactic treatment. Treatment may also include arresting progression in the severity of a disease.

The term “treat”, “treating” or “treatment” of any disease or disorder refers in one embodiment, to ameliorating the disease or disorder (i.e., slowing or arresting or reducing the development of the disease or at least one of the clinical symptoms thereof). In another embodiment “treat”, “treating” or “treatment” refers to alleviating or ameliorating at least one physical parameter including those which may not be discernible by the patient. In yet another embodiment, “treat”, “treating” or “treatment” refers to modulating the disease or disorder, either physically, (e.g., stabilization of a discernible symptom), physiologically, (e.g., stabilization of a physical parameter), or both. In yet another embodiment, “treat”, “treating” or “treatment” refers to preventing or delaying the onset or development or progression of the disease or disorder. As used herein, a subject is “in need of a treatment if such subject would benefit biologically, medically or in quality of life from such treatment.

Subject

The term “subject” means any animal, including humans and companion animals. Generally, the subject is a human or an avian, bovine, canine, equine, feline, hircine, murine, ovine or porcine animal. The subject can be a horse or a companion animal, for example a cat or a dog. Preferably, the subject is a human.

The treatment of mammals, particularly humans, is preferred. However, both human and veterinary treatments are within the scope of the invention.

For veterinary subjects, dogs, cats and equine subjects are preferred.

The present invention may also be useful in non-human animal subjects such as: avian, bovine, ovine or porcine animals, for optimizing meat production by increasing skeletal muscle mass and/or function.

Muscle Stem Cells

The term “muscle stem cell”, as used herein, may refer to satellite cells, preferably satellite cells that are quiescent and are uncommitted.

Satellite cells are precursors to skeletal muscle cells. In adult muscle, satellite cells are generally quiescent, but can activate and undergo myogenesis in response to disease or mechanical strain such as injury or exercise. Satellite cells are also involved in the normal growth of muscle. Upon activation, satellite cells proliferate before undergoing myogenic differentiation to finally fuse with existing myofibers or to form new myofibers, depending on the magnitude of tissue trauma. In addition to generating differentiated myogenic progeny, at least some satellite cells can self-renew, thereby meeting the defining criteria of bona fide resident stem cells.

Pax7 is the most well-known and characterized marker express by muscle stem cells i.e. muscle stem cells can be reliably identified based on their expression of the paired box transcription factor Pax7. The muscle stem cells may also express NCAM, CD56, CD29 and/or CD82, i.e. the muscle stem cells may be NCAM+, CD56+, CD29+ and/or CD82+.

MyoD+ is a commitment marker that may be used to distinguish quiescent from committed satellite cells.

Muscle Function and Mass

The compounds, compositions, uses and methods disclosed herein may provide for the maintenance of or increase in muscle function and/or mass.

The term “muscle function” refers to the ability of a muscle to perform in a manner that does not negatively impact on the life of a subject, and encompasses parameters of muscle strength, muscle contraction, muscle endurance, muscle elasticity, ability of a muscle to resist muscle fatigue and/or physical activities of daily living such as walking up stairs, getting out of a chair and other activities of daily living.

Suitable tests for assessing muscle function include grip strength assessment using a dynamometer; one repeat maximum on leg press, chest press or leg extension; gait speed; 6 min walk test; time up and go; short physical performance battery; Fried frailty criteria; and stair climbing time assessments. Other suitable tests include muscle strength, endurance and time to fatigue.

Muscle mass (which may equate with muscle volume, muscle thickness or myofiber size) may be measured by dual-energy X-ray absorptiometry (DXA) or bioimpedance tests. Similarly, MRI may be used for assessing muscle volume and ultra-sound may be used for assessing muscle thickness and pennation angle.

“Muscle wasting” may be a reduction in muscle mass, for example to the stage where the muscle loss becomes debilitating. In one embodiment, the subject does not lose more than 10%, 5%, 4%, 3%, 2% or 1% of their muscle mass.

Preferably, the compounds, compositions, uses and methods disclosed herein provide for the maintenance of or increase in muscle mass.

The term “maintains” refers to a particular parameter, such as muscle function and/or mass, remaining substantially unchanged over a period of time (e.g. 5, 10, 15, 20, 25, 30, 40, 50 or more years).

In one embodiment, muscle mass increases by at least 1%, 2%, 3%, 4%, 5%, 10%, 15% or 20%.

In another embodiment, muscle mass increases by 1-2.5%, 1-5%, 1-10% or 1-20%.

Preferably, the muscle is skeletal muscle.

### EXAMPLES

### Example 1: Selection of Compounds Modulating Muscle Stem Cells

Selection of Human Skeletal Muscle Myoblasts

The inventors developed a high content screening to test in vitro compounds on human primary adult muscle cells. Human Skeletal Muscle Myoblasts (HSMM) were purchased from Lonza (https://bioscience.lonza.com). These cells were isolated from the upper arm or leg muscle tissue of normal donors and used after the second passage. Several donors were tested to ensure cell viability and purity before selecting the final donors, which are a 36-year-old Caucasian female (Donor 8) and a 20-year-old Caucasian female (Donor 4).

Assay for Muscle Stem Cell Commitment

The primary screening assay was based on the high content detection of two important myogenic regulatory factors (Pax7 and MyoD) by immunofluorescence. Pax7 and MyoD are the major hallmarks of muscle stem cell stemness and commitment and can be used to monitor muscle stem cell progeny. In particular, Pax7 marks early amplification while MyoD is a later marker for myogenic commitment, and combinations of these markers define the different states of proliferation, differentiation and self-renewal.

The hit selection was primarily based on compounds that can enhance the commitment toward the myogenic differentiation (Pax7-/MyoD+ cells), which is particularly relevant in the context of cancer cachexia where a defect in myogenic commitment has been revealed as a potential cause of the muscle wasting (He et al. 2013).

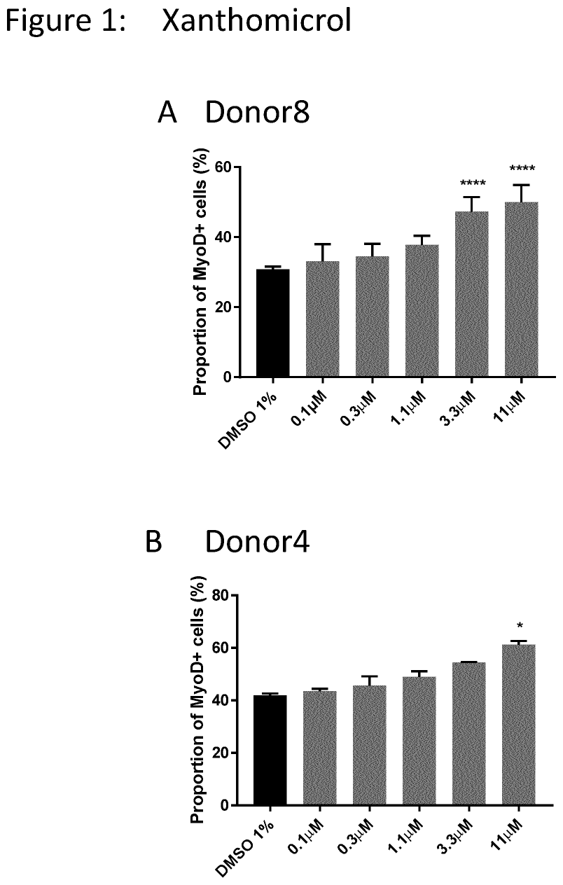

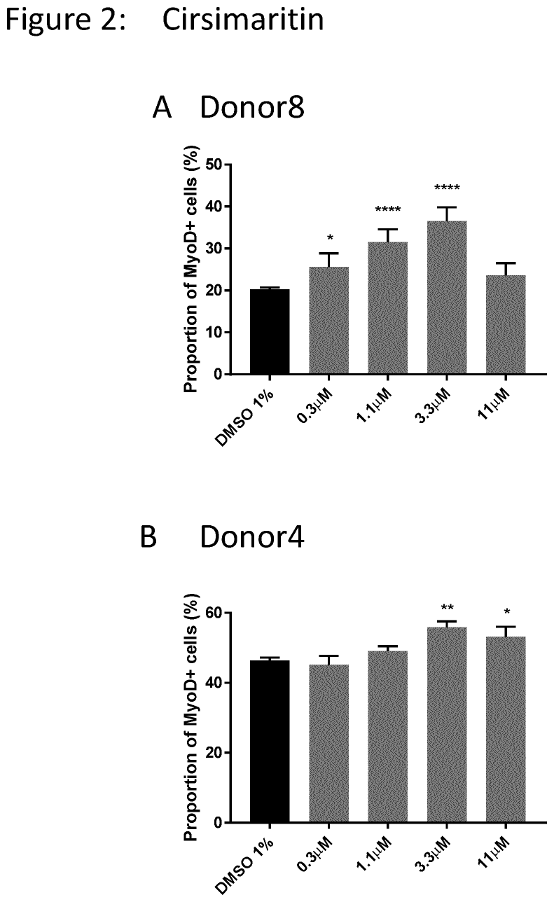

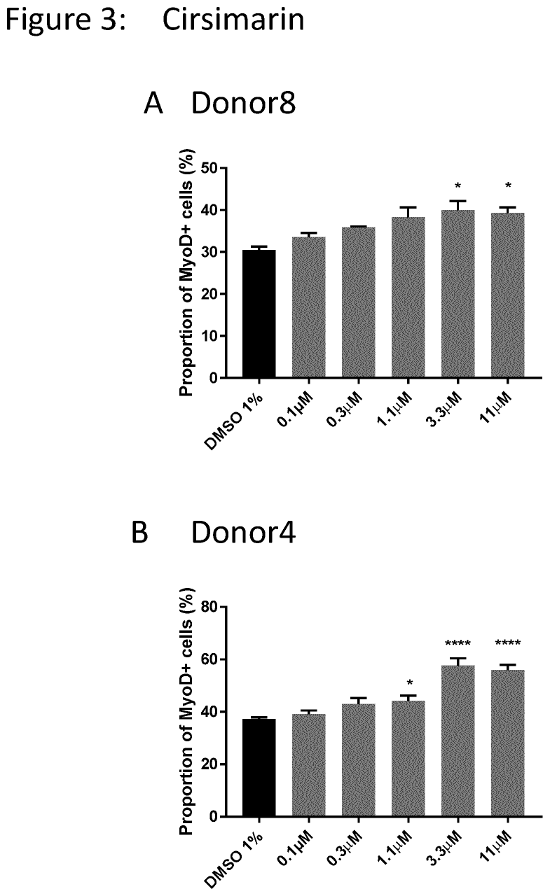

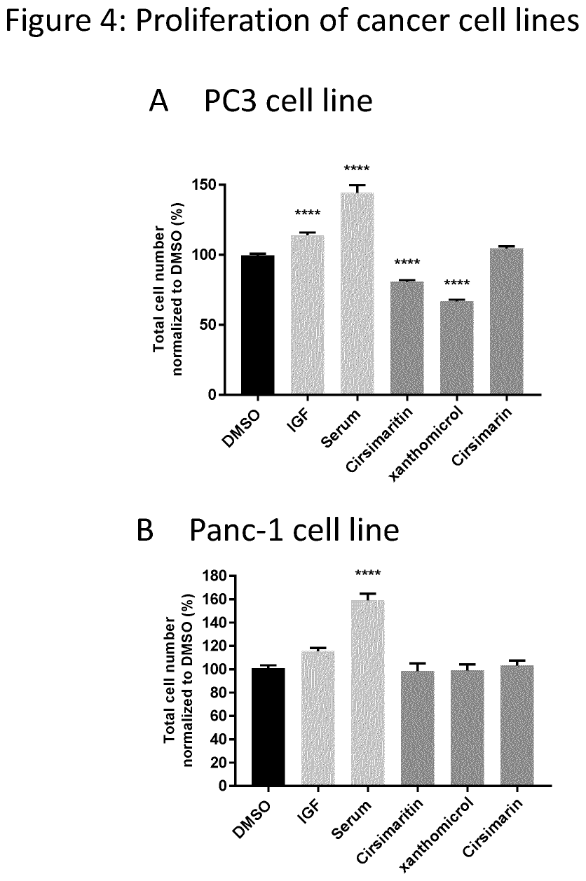

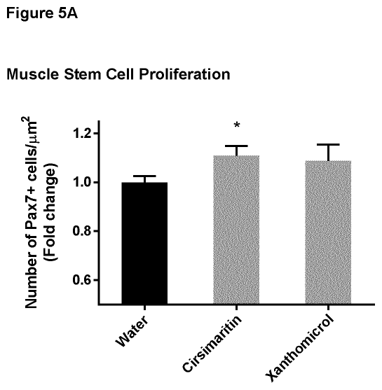

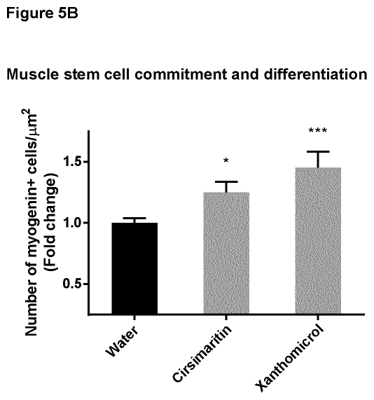

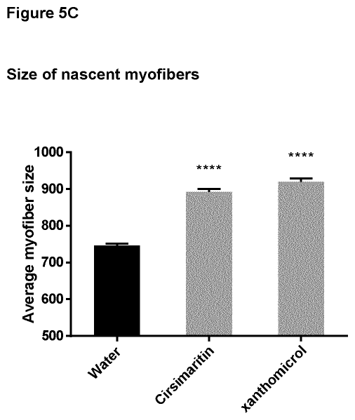

Human primary myoblasts were seeded in 384 well plates at a density of 1′000 cells per well in skeletal muscle growth medium (SKM-M, AMSbio). For treatment, compounds were directly added to the myoblast cultures 16 hours after initial plating. All cultures were then grown for 96 hours. Cells were stained for Pax7 and MyoD expression using antibodies directed against Pax7 and MyoD and counterstained with Hoechst 33342 to visualize cell nuclei. MyoD+ cells are defined as cells that do not express Pax7 but express MyoD. Pax7+ cells are defined as cells that express Pax7 regardless of MyoD expression. Image acquisition was performed using the ImageXpress (Molecular Devices) platform. Custom module analysis based on Multi-Wavelength Cell Scoring of the MetaXpress software was used for quantification. FIGS. 1 to 3 show the results for each of the preferred compounds: xanthomicrol, cirsimaritin and cirsimarin, respectively, with MyoD+ cells normalized to the total cell number in order to evaluate the myogenic commitment.

### Example 2: In Vivo Muscle Regeneration Using Cirsimaritin or Xanthomicrol

In order to reproduce the physiological process of muscle regeneration that occurs in adult skeletal muscles in response to injury or disease, we performed an intramuscular injection of cardiotoxin into mouse hindlimb muscles. One week prior to the induction of the muscle injury, mice were given by oral gavage compounds of the invention; either cirsimaritin or xanthomicrol at 100 mg/kg body weight and water was used as a control. Mice were treated once a day until the end of the experiment. To evaluate the efficiency of the muscle regeneration, muscles that have been previously injured were harvested 5 days after the injury and cryosections were prepared. Several myogenic markers were then measured. Cryosections were stained for Pax7, Myogenin, laminin and embryonic Myosin Heavy Chain (eMHC) expression using specific antibodies and counterstained with Hoechst 33342 to visualize cell nuclei.

FIG. 5 demonstrates that cirsimaritin and xanthomicrol were each able to promote the in vivo muscle regeneration process. In particular, this is shown be the early phase of expansion evaluated by counting the number of Pax7+ cells shown in FIG. 5A and the subsequent phase of myogenic differentiation of Muscle Stem Cells measured by counting the number of myogenin+ cells shown in FIG. 5B, respectively. Data were expressed as number of cells per arear of injured muscle. In addition, the size of each newly formed myofiber after administration of cirsimaritin or xanthomicrol compared to the control (water) was measured based on the expression of the eMHC and laminin that allowed recognition and delineation of these nascent myofibers. Results are shown as mean myofiber cross-sectional area (μm²).

In all FIGS. 5A, B and C *, **, ***, **** indicates difference from the control, One-way ANOVA, with p<0.05, p<0.01, p<0.001, p<0.0001, respectively. Data are presented as Mean+/−SEM.

### REFERENCES

Almeida et al. (2016) Muscle Satellite Cells: Exploring the Basic Biology to Rule Them, Stem Cells International, Vol. 2016, ID 1078686.
Chen, L. K., et al. (2014). Sarcopenia in Asia: consensus report of the Asian Working Group for Sarcopenia. Journal of the American Medical Directors Association 15, 95-101.
Clark R V, Walker A C, O'Connor-Semmes R L, Leonard M S, Miller R R, Stimpson S A, Turner S M, Ravussin E, Cefalu W T, Hellerstein M K, Evans W J (1985). Total body skeletal muscle mass: estimation by creatine (methyl-d3) dilution in humans. J Appl Physiol. June 15; 116(12):1605-13.
Cruz-Jentoft, A. J., Baeyens, J. P., Bauer, J. M., Boirie, Y., Cederholm, T., Landi, F., Martin, F. C., Michel, J. P., Rolland, Y., Schneider, S. M., et al. (2010). Sarcopenia: European consensus on definition and diagnosis: Report of the European Working Group on Sarcopenia in Older People. Age Ageing 39, 412-423.
Fearon et al. (2011) Definition and classification of cancer cachexia: an international consensus. Lancet Oncology, 12, 489-495.
He W A, Berardi E, Cardillo V M, Acharyya S, Aulino P, Thomas-Ahner J, Wang J, Bloomston M, Muscarella P, Nau P, Shah N, Butchbach M E, Ladner K, Adamo S, Rudnicki M A, Keller C, Coletti D, Montanaro F, Guttridge D C (2013). NF-κB-mediated Pax7 dysregulation in the muscle microenvironment promotes cancer cachexia. J Clin Invest. November; 123(11):4821-35.
Studenski S A, Peters K W, Alley D E, Cawthon P M, McLean R R, Harris T B, Ferrucci L, Guralnik J M, Fragala M S, Kenny A M, Kiel D P, Kritchevsky S B, Shardell M D, Dam T T, Vassileva M T (2014). The FNIH sarcopenia project: rationale, study description, conference recommendations, and final estimates. J Gerontol A Biol Sci Med Sci. 69(5), 547-558.
Stimpson S A, Leonard M S, Clifton L G, Poole J C, Turner S M, Shearer T W, Remlinger K S, Clark R V, Hellerstein M K, Evans W J. (2013) Longitudinal changes in total body creatine pool size and skeletal muscle mass using the D<sub>3</sub>-creatine dilution method. J Cachexia Sarcopenia Muscle. June 25.
von Haehling, S. and S. D. Anker, Prevalence, incidence and clinical impact of cachexia: facts and numbers-update (2014). J Cachexia Sarcopenia Muscle, 5(4): p. 261-3 (2014).

1. A method to maintain or increase skeletal muscle function and/or mass in a subject, and/or substantially prevent or reduce muscle wasting in a subject comprising administering to the subject in need of same a compound represented by formula (I): 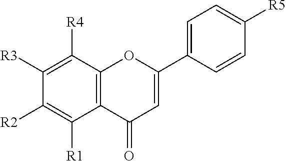
wherein R1, R2, R3, and R4, are each independently selected from the group consisting of H; OH; OMe; O-glycoside; C-glycoside; acylated O-glycoside; acylated C-glycoside; sulfated O-glycoside; sulfated C-glycoside; a halogen; a ketone; an aldehyde; a carboxylic acid; an ester; a primary, secondary, or tertiary amine; a primary or secondary amide; a cyano; a nitro; a sulfonate; and a sulfate; and R5 is selected from the group consisting of H; OH; O-glycoside; C-glycoside; acylated O-glycoside; acylated C-glycoside; sulfated O-glycoside; sulfated C-glycoside; a halogen; a ketone; an aldehyde; a carboxylic acid; an ester; a primary, secondary, or tertiary amine; a primary or secondary amide; a cyano; a nitro; a sulfonate; and a sulfate.

2. A method according to claim 1 comprising administering to the subject a compound represented by formula (II): 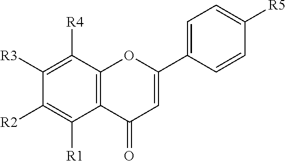
wherein R1, R2, R3, and R4 are each independently selected from the group consisting of H; OH; OMe; O-glycoside; a sulfate; a substituted and branched C1 to C20 alkyl; a substituted and branched, C2 to C20 alkenyl; a substituted and branched, C4 to C20 polyalkenyl and R5 is selected from the group consisting of H; OH; O-glycoside; a sulfate; a substituted and branched C1 to C20 alkyl; a substituted and branched, C2 to C20 alkenyl; a substituted and branched, C4 to C20 polyalkenyl.

3. A method according to claim 1 to maintain or increase skeletal muscle function and/or mass in a subject, and/or substantially prevent or reduce muscle wasting in a subject by administering a compound represented by formula (III): 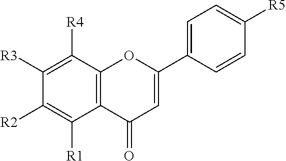
wherein R1, R2, R3, and R4 are each independently selected from the group consisting of H; OH; OMe; O-glycoside; or a sulfate and R5 is H; OH; O-glycoside; and a sulfate.

4. A compound according to claim 1 wherein the compound is selected from the group consisting of:
xanthomicrol (5,4′-Dihydroxy-6,7,8-trimethoxyflavone, CAS number 16545-23-6), 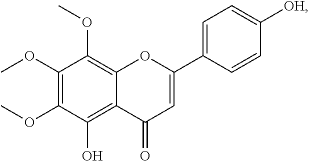 [SMILES: COc1c(OC)c(O)c2c(=O)cc(-c3ccc(O)cc3)oc2c1OC]
cirsimaritin (5,4′-Dihydroxy-6,7-dimethoxyflavone, CAS number 6601-62-3) 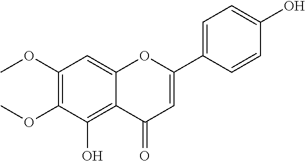 [SMILES: COc1cc2oc(-c3ccc(O)cc3)cc(=O)c2c(O)c1OC]
and
cirsimarin (5,4′-Dihydroxy-6,7-dimethoxyflavone 4′-O-β-D-glucoside, CAS number 13020-19-4).

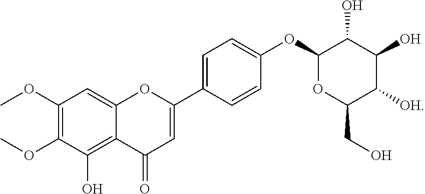 [SMILES: COc1cc2oc(-c3ccc(O[C@@H]4O[C@H](CO)[C@@H](O)[C@H](O)[C@H]4O)cc3)cc(=O)c2c(O)c1OC]

5. A method to maintain or increase skeletal muscle function and/or mass in a subject, and/or substantially prevent or reduce muscle wasting in a subject in need of same comprising administering to the subject a compound represented by formula (IV): 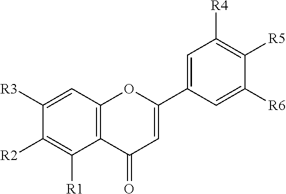
wherein R1, R2, and R3 are each independently selected from the group consisting of H; OH; OMe; O-glycoside; C-glycoside; acylated O-glycoside; acylated C-glycoside; sulfated O-glycoside; sulfated C-glycoside; a halogen; a ketone; an aldehyde; a carboxylic acid; an ester; a primary, secondary, or tertiary amine; a primary or secondary amide; a cyano; a nitro; a sulfonate; a sulfate; a substituted and branched C1 to C20 alkyl; a substituted and branched, C2 to C20 alkenyl; a substituted and branched, C4 to C20 polyalkenyl; a substituted and branched C2 to C20 alkynyl, and a substituted and branched C4 to C20 polyalkynyl and R4, R5 and R6, are each independently selected from the group consisting of OH; OMe; O-glycoside; C-glycoside; acylated O-glycoside; acylated C-glycoside; sulfated O-glycoside; sulfated C-glycoside; a halogen; a ketone; an aldehyde; a carboxylic acid; an ester; a primary, secondary, or tertiary amine; a primary or secondary amide; a cyano; a nitro; a sulfonate; a sulfate; a substituted and branched C1 to C20 alkyl; a substituted and branched, C2 to C20 alkenyl; a substituted and branched, C4 to C20 polyalkenyl; a substituted and branched C2 to C20 alkynyl, and a substituted and branched C4 to C20 polyalkynyl.

6. A method according to claim 5 to maintain or increase skeletal muscle function and/or mass in a subject, and/or substantially prevent or reduce muscle wasting in a subject comprising administering a compound represented by formula (V): 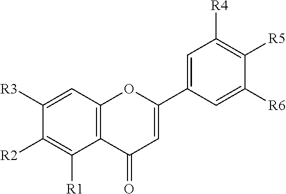
wherein R1, R2 and R3 are each independently selected from the group consisting of H; OH; OMe; O-glycoside; a sulfate; a substituted and branched C1 to C20 alkyl; a substituted and branched, C2 to C20 alkenyl; and a substituted and branched, C4 to C20 polyalkenyl and R4, R5 and R6, are each independently selected from the group consisting of OH; OMe; O-glycoside; a sulfate; a substituted and branched C1 to C20 alkyl; a substituted and branched, C2 to C20 alkenyl; and a substituted and optionally branched, C4 to C20 polyalkenyl.

7. A method according to claim 5 to maintain or increase skeletal muscle function and/or mass in a subject, and/or substantially prevent or reduce muscle wasting in a subject comprising administering a compound represented by formula (VI): 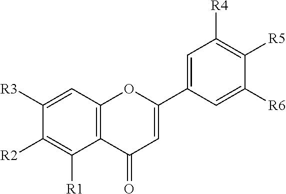
wherein R1, R2 and R3 are each independently selected from the group consisting of H; OH; OMe; O-glycoside; a sulfate and R4, R5 and R6, are each independently OH; OMe; O-glycoside; a sulfate.

8. A method according to claim 5 wherein the compound 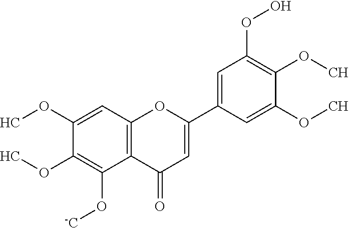
is 5,6,7,3′,4′,5′-hexamethoxyflavone, CAS number 29043-07-0.

9. A method to maintain or increase skeletal muscle function and/or mass in a subject, and/or substantially prevent or reduce muscle wasting in a subject in need thereof comprising administering to the subject a compound represented by formula (VII): 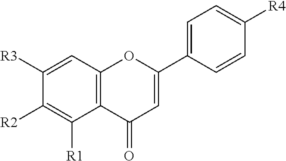
wherein R1 and R2 are each independently H or OH and R3 and R4, are each independently H; OH; OMe; O-glycoside; C-glycoside; acylated O-glycoside; acylated C-glycoside; sulfated O-glycoside; sulfated C-glycoside; a halogen; a ketone; an aldehyde; a carboxylic acid; an ester; a primary, secondary, or tertiary amine; a primary or secondary amide; a cyano; a nitro; a sulfonate; a sulfate; a substituted and branched C1 to C20 alkyl; a substituted and branched, C2 to C20 alkenyl; a substituted and branched, C4 to C20 polyalkenyl; a substituted and branched C2 to C20 alkynyl, and a substituted and branched C4 to C20 polyalkynyl.

10. A method according to claim 9 to maintain or increase skeletal muscle function and/or mass in a subject, and/or substantially prevent or reduce muscle wasting in a subject comprising administering to the subject a compound represented by formula (VIII): 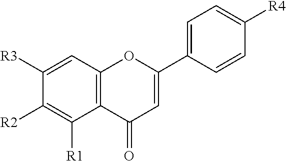
wherein R1 and R2 are each independently selected from the group consisting of H or OH and R3 and R4, are each independently H; OH; OMe; O-glycoside; a sulfate; a substituted and branched C1 to C20 alkyl; a substituted and branched, C2 to C20 alkenyl; and a substituted and branched, C4 to C20 polyalkenyl.

11. A method according to claim 9 to maintain or increase skeletal muscle function and/or mass in a subject, and/or substantially prevent or reduce muscle wasting in a subject comprising administering a compound represented by formula (IX): 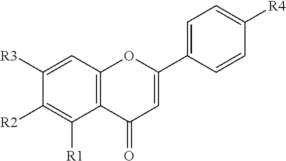
wherein R1 and R2 are each independently H or OH and R3 and R4, are each independently selected from the group consisting of H; OH; OMe; O-glycoside; and a sulfate.

12. A method according to claim 9 wherein said compound 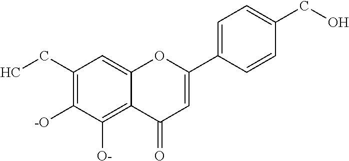
is ladanein (5,6-Dihydroxy-7,4′-dimethoxyflavone, CAS number 10176-71-3).

13-31. (canceled)
